(19) United States
(12) Patent Application Publication
(10) Pub. No.: US 2005/0106584 A1
(43) Pub. Date: May 19, 2005

(54) Alu-derived exons and uses thereof for detection and treatment of genetic diseases

(76) Inventors: Gil Ast, Ramat Aviv (IL); Galit Lev-Maor, Tel-Aviv (IL); Rotem Sorek, Tel Aviv (IL); Noam Shomron, Herzelia (IL)

Correspondence Address: WINSTON & STRAWN;PATENT DEPARTMENT, 1400 L STREET, N.W., WASHINGTON, DC, 20005-3502, US

(21) Appl. No.: 10/853,641

(22) Filed: May 24, 2004

Related U.S. Application Data
(60) Provisional application No. 60/526,599, filed on Dec. 4, 2003.

(30) Foreign Application Priority Data
May 22, 2003 (IL) IL 156072

(51) Int. Cl. C12Q 1/68; C07H 21/04; C12N 15/63

(52) U.S. Cl. 435/6; 435/320.1; 536/23.5; 536/24.3

(57) ABSTRACT

The present invention relates to polynucleotides comprising Alu-derived exons derived by alternative splicing from Alu elements, which are involved in cancer and genetic diseases. The present invention further relates to use of oligonucleotides for identification of the sequences associated with Alu exonization for diagnosis and treatment of cancer and genetic diseases.

This application claims the benefit of provisional application 60/526,599 filed Dec. 4, 2003, the entire content of which is expressly incorporated herein by reference thereto.

### FIELD OF THE INVENTION

The present invention relates to polynucleotides comprising Alu-derived exons derived by alternative splicing from Alu elements, which are involved in cancer and genetic diseases. The present invention further relates to use of oligonucleotides for identification of the sequences associated with Alu exonization for diagnosis and treatment of cancer and genetic diseases.

### BACKGROUND OF THE INVENTION

Alu elements are short, interspersed elements (SINEs) about 300 nucleotides in length, which amplify in primate genomes through a process of retroposition. Alu elements represent a significant fraction of noncoding DNA.

Numerous studies describe splicing-mediated insertions of part of Alu sequence into mature mRNAs, a process known as exonization of Alu elements. It was recently described by some of the applicants of the present invention that 5.2% of the alternatively spliced exons observed in public mRNA and EST databases originate from Alu sequence, whereas none of the constitutively spliced exons are Alu-derived (Sorek et al., Genome Res., 12:1060-1067, 2002). The results indicate that internal exons containing an Alu sequence are predominantly, if not exclusively, alternatively spliced. Sorek et al. postulated that the absence or dearth of constitutive Alu-containing exons in the human genome indicates the negative selection of such exons, which probably interfere with normal protein production.

A study published after the priority date of the present application by the inventors of the present invention discloses that two positions along the inverted Alu sequence are most commonly used as a 3′ splice-site in Alu exonization (Lev-Maor et al., Science 300:1288-1291, July 2003). Another study published after the priority date of the present application by the inventors of the present invention discloses that certain mutational changes are required in order to create functional 5′splice-sites in Alu elements (Sorek et al., Molecular Cell, 14:221-231, April 2004).

A putative role for an Alu upstream motif or cis element in glaucoma pathogenesis is disclosed in U.S. Pat. No. 6,171,788. This patent discloses that transcription of Trabecular Meshwork Inducible Glucocorticoid Response (TIGR) promoter molecules can be effected by agents capable of altering the biochemical properties or concentration of nuclear factors or their homologues, including nuclear factors bound to Alu elements. Such agents can be used in the study and treatment of glaucoma.

Association of Alu introns with hereditary types of cancer is disclosed in U.S. Pat. No. 6,733,966. This patent discloses unusual high concentration of Alu-elements in the BRCA1 gene intronic regions favoring the induction of large genomic deletions and inversions in a situation of increased genomic instability.

Predisposition to genetic diseases determined by detecting mutations in DNA sequences is disclosed in numerous publications. For example, U.S. Pat. No. 5,849,483 discloses a high-throughput method for screening nucleic acid samples to identify target sequences or genetic alterations in target sequences present in the nucleic acid samples including randomly permuted alterations in nucleic acid sequences of interest. Methods of detecting mutations in DNA sequences using a competitive oligonucleotide priming system is disclosed in U.S. Pat. No. 6,015,675. Mass spectrometry-based processes for detecting particular nucleic acid molecules and sequences in the molecules are disclosed in U.S. Pat. No. 6,258,538. Depending upon the sequence to be detected, the processes can be used to diagnose a genetic disease or a chromosomal abnormality, a predisposition to a disease or condition, or infection by a pathogen, or for determining identity or heredity.

A variety of ‘wet laboratory’ techniques for identification of specific genetic sequences or genetic markers are known in the art. For example, U.S. Pat. No. 6,528,256 discloses a method for the identification and isolation of specific genetic sequences or genetic markers of cDNA or of genomic DNA derived from the cells, tissues or organs of an organism, particularly a mammal, employing an Amplification Fragment Length Polymorphism (AFLP)-based technique. AFLP is disclosed in EP 0 534 858 and is PCR-based with specific combinations of restriction endonucleases and adapters of discrete sequences, as well as primers that contain the common sequences of the adapters. In this way, a sequence or fragment of DNA in a complex sample may be specifically amplified and used for further analysis.

Identification of specific genetic sequences or genetic markers can be also effected by computational algorithms, by mining data from existing databases.

Treating genetic diseases by preventing expression of undesired genetic elements is known in the art. U.S. Pat. No. 5,681,747 discloses methods for inhibiting human-PKCα expression with an oligonucleotide specifically hybridizable to a portion of the 3′-untranslated region of PKCα. A method for gene therapy using small fragment homologous replacement is disclosed in U.S. Pat. No. 6,010,908. The method introduces small fragments of exogenous DNA into regions of endogenous genomic DNA virtually homologous to the exogenous DNA. The exogenous DNA fragment contains sequence modification that correct mutations in the endogenous DNA or introduce mutations that alter cellular or an infecting pathogen phenotype. U.S. Pat. No. 6,506,559 discloses methods for inhibiting gene expression in vitro using certain isiRNA constructs that mediate RNAi. A strategy for suppressing expression of an endogenous gene comprising providing suppression effectors able to bind to the non-coding regions of a gene to be suppressed, to prevent the functional expression thereof, is disclosed in U.S. Pat. No. 6,713,457. The suppression effectors may be antisense nucleic acids, and the non-coding regions can include the transcribed but non-translated regions of a gene. The strategy can also introduce a replacement gene. International Publication No. WO 99/53050 discloses certain methods for decreasing the phenotypic expression of a nucleic acid in plant cells using certain double strand RNAs. International Publication No. WO 01/49844 discloses specific DNA constructs for use in facilitating gene silencing in targeted organisms. International Publications Nos. WO 02/055692, WO02/055693, and EP 1144623 B1 disclose methods for inhibiting gene expression using RNAi. International Publications Nos. WO 99/49029 and WO01/70949, and AU 4037501 describe certain vector expressed siRNA molecules.

Nowhere in the background art it is taught or suggested that Alu-derived silent sequences may be transformed into exonic sequences and that such exonized Alu-derived sequences may be involved in the pathogenesis of genetic disease.

### SUMMARY OF THE INVENTION

The present invention relates generally to identification and use of genetic elements that are hitherto uncharacterized markers of diseases or predisposition to genetic diseases. In particular, the present invention relates to genetic markers comprising Alu-derived exons derived from Alu introns.

It is now disclosed that the exonization of mutated Alu sequences is a factor that may be linked to the loss or alteration of normal gene function. The detection of exons derived from Alu introns is now disclosed to provide novel markers useful for prognosis, diagnosis or treatment of a disease or condition selected from the group consisting of a genetic disease, a chromosomal abnormality, a genetic predisposition, and a viral infection.

The present invention is based in part on the unexpected discovery that Alu elements or fragments thereof, predominantly in their antisense orientation inserted into mature mRNAs by way of splicing (‘exonization’), are associated with genetic disease and cancer.

Thus, the present invention relates to method of identifying, inhibiting and treating diseases, including but not limited to genetic diseases and cancer, utilizing Alu-derived genetic markers. The Alu-derived genetic markers of the invention can be used for detection or treatment of a disease and can be also used for identifying alternatively spliced mRNA isoforms containing same that are prevalent in the disease, thereby enabling use of the mRNA isoform for detection or treatment of said disease. The first step of the latter approach can be conducted in silico, using well known EST, cDNA and mRNA libraries for the identification of alternatively spliced mRNA isoforms containing Alu-derived exons.

According to one aspect the present invention provides an isolated polynucleotide comprising at least one Alu-derived exon comprising a sequence derived by alternative splicing of an Alu element, the Alu element comprising at least one mutated splice site. According to particular embodiments the mutated splice site is as set forth in any one of SEQ ID NOS:1-58.

According to one embodiment, the sequence comprising the mutated splice site of the Alu element is at least 80% homologous, preferably 85%, more preferably 90%, most preferably 95% or more homologous to any one of SEQ ID NOS:1-58.

Without wishing to be bound by any particular theory or mechanism of action, Alu introns transform into Alu exons upon evolution of certain mutations within pseudo splice-sites (SS) in the intronic Alu sequences, a process referred to herein as exonization.

Surprisingly, the inventors of the present invention discovered particular positions along Alu sequences that are most commonly used as splice-sites in Alu exonization. Based on these findings the inventors of the present invention identified 7,810 Alu sequences within EST libraries (Sorek et al. 2004, ibid).

According to yet another embodiment, the isolated polynucleotides of the present invention comprise alternative splice-sites, also referred herein as pseudo splice-sites. According to yet another embodiment, the polynucleotides of the present invention comprise sequences downstream to the 3′ splice-site that is involved in Alu exonization. According to certain embodiments, the 3′ splice-site is within positions 275 to 279 of the consensus Alu sequence, corresponding to positions 12 to 16 of SEQ ID NOS:1-32. According to yet another embodiment, the polynucleotides of the present invention comprise sequences upstream to the 5′ splice-site that is involved in Alu exonization. According to certain embodiments, the 5′ splice-site is in positions 154 to 160 within the consensus Alu sequence, corresponding to positions 13 to 19 of SEQ ID NOS: 33-58.

It is now disclosed for the first time that that out of the at least 238,000 antisense Alu sequences located within introns in the human genome 52,935 Alus carry a potential ADAR2-like 3′SS (Slavov et al., GENE, 299:83, 2002) and 23,012 carry a potential PGT-like 3′SS (NCBI GenBank Access No.: AA225691) suggesting that many of these silent intronic Alu elements might be susceptible to exonization by the same single point mutation, and are thus under strict selective pressure. Such point mutations in human genomic antisense Alu sequences may be the molecular basis for predisposition to previously uncharacterized genetic diseases.

According to another aspect the present invention provides an oligonucleotide comprising at least a portion of an Alu-derived exon selected from an oligonucleotide probe and an oligonucleotide primer, the Alu-derived exon comprising a sequence derived by alternative splicing of an Alu element, the Alu element comprising at least one mutated splice site. According to particular embodiments the mutated splice site is as set forth in any one of SEQ ID NOS:1-58.

According to one embodiment, said oligonucleotide further comprises a detectable label. The labels include, but are not limited to, fluorophores, chromophores, radioactive isotopes, electron dense reagents, enzymes, enzymatic substrates and ligands.

It is to be understood explicitly that the scope of the present invention encompasses homologs, analogs, variants and derivatives, including but not limited to shorter and longer polynucleotides or oligonucleotides, with one or more nucleic acid substitution, as well as nucleic acid derivatives, non-natural nucleic acids and synthetic nucleic acids as are known in the art.

According to one embodiment, the present invention provides a vector comprising the isolated polynucleotide of the invention. According to another embodiment, the present invention provides a vector comprising the oligonucleotide of the invention. According to yet another embodiment, the present invention provides a construct comprising the oligonucleotide of the invention.

According to yet another aspect, the present invention provides a method for diagnosing, detecting or predisposition to a disease, comprising determining the presence in a biological specimen of a polynucleotide comprising a sequence selected from: a) an Alu-derived sequence comprising at least one mutated alternative splice site; and b) an alternatively spliced mRNA isoform comprising an Alu-derived exon.

According to one embodiment, the Alu-derived sequence of a) comprises a sequence as set forth in any one of SEQ ID NOS: 1-58, fragments or extensions thereof.

According to another embodiment, the disease is selected from the group consisting of: cancer, a genetic disease, a chromosomal abnormality, a genetic predisposition and a viral infection. According to another embodiment, the genetic disease is selected from the group consisting of: Alport syndrome, Sly syndrome, CCFDN (congenital cataracts, facial dysmorphism, and neuropathy) syndrome and OAT deficiency.

According to yet another embodiment, cancer is selected from the group consisting of: prostate cancer, breast cancer, ovarian cancer, lung cancer, melanoma, renal cancer, bladder cancer, fibrosarcoma, hepatocellular carcinoma, osteocarcinoma, primary ductal carcinoma, giant cell sarcoma, ductal carcinoma, Hodgkin's disease, colorectal carcinoma, lymphoma, transitional cell carcinoma, uterine sarcoma, adenocarcinoma, plasmacytoma, epidermoid carcinoma, Burkitt's lymphoma, Ewing's sarcoma, gastric carcinoma, squamous cell carcinoma, neuroblastoma and rhabdomyosarcoma.

According to one embodiment, the method comprises:
(a) obtaining a biological specimen;
(b) contacting the biological specimen with a nucleic acid probe capable of hybridizing specifically the polynucleotide of the invention or fragments thereof, under conditions suitable for hybridization; and
(c) determining the presence of a hybrid in said biological specimen.

According to yet another embodiment, the biological specimen is derived from a human or non-human primate. According to yet another embodiment, the biological specimen is selected from a tissue sample, fluids, cells, cellular extract, tissue extract and genetic material extracted from same, including but not limited to DNA and RNA. According to yet another embodiment, the fluids are selected from the group consisting of: blood, urine and saliva.

According to another embodiment, the method further comprises:
(a) generating a nucleic acid probe capable of hybridizing specifically with an alternatively spliced mRNA isoform or fragments thereof;
(b) obtaining a biological specimen;
(c) contacting the biological specimen with the nucleic acid probe under conditions suitable for hybridization; and
(d) determining the presence of a hybrid in said biological specimen.

According to one embodiment, the method comprises identifying the alternatively spliced mRNA isoforms in silico from a database of sequences selected from the group consisting of: cDNA, EST, and mRNA.

According to another embodiment, the alternatively spliced mRNA isoform is identified by a method selected from a group consisting of: Northern blot analysis, RNase protection, in situ hybridization and selective hybridization to arrayed cDNA libraries.

According to yet another embodiment, the method further comprises contacting said biological specimen with a control nucleic acid probe capable of forming a hybrid with at least one control gene or fragments thereof. According to yet another embodiment, the at least one control gene is selected from the group consisting of: β-actin, glycer-aldehyde-phophate-dehydrogenase (GAPDH), S16 rRNA, phosphoribosyl transferase (HPRT), dihydrofolate reductase (DHFR), adenosine deaminase, phosphoglycerol kinase (PGK), pyruvate kinase, phosphoglycerol mutase.

According to yet another embodiment, the method further comprises contacting the nucleic acid probe with a biological standard. According to certain embodiments, the biological standard is derived from ostensibly healthy tissue of a human or a non-human primate and said biological specimen is derived from a non-healthy tissue derived from the human or the non-human primate. Alternatively, said biological specimen is derived from a non-healthy tissue derived from a different human or non-human primate.

According to yet another embodiment, the nucleic acid probe is labeled. According to certain embodiments, the labels are selected from fluorophores, chromophores, radioactive isotopes, electron dense reagents, enzymes, and ligands having specific binding partners.

According to yet another embodiment, the method comprising:
(a) obtaining a genetic material derived from a biological specimen;
(b) contacting the genetic material with a reaction mixture comprising a plurality of primers that are capable of amplifying the polynucleotide of the invention or fragments thereof, by polymerase chain reaction; and
(c) separating the amplified products.

According to yet another embodiment, the plurality of primers comprises at least one primer that is labeled to enable detection of the amplified products. According to some alternative embodiments, the amplified products are separated by means of molecular size exclusion.

According to yet another embodiment, the reaction mixture comprises the four nucleoside triphosphates: dATP, dCTP, dTTP, and dGTP. According to an alternative embodiment, the reaction mixture comprises dATP, dCTP, dTTP, dGTP, and an analog of dGTP, including but not limited to, inosine, 7-deaza-guanosine, 7-deaza inosine deoxyribonucleotides and 2′-deoxy analogs thereof.

According to another embodiment, the genetic material is extracted from tissue, cells and body fluids. According to yet another embodiment, the fluids are selected from the group consisting of: blood, sperm, urine and saliva.

According to a preferred embodiment, the alternatively spliced mRNA isoform is an ACAD-9 mRNA (Zhang et al., Biochem. Biophys. Res. Commun., 4:297:1033, 2002). According another preferred embodiment, the disease is ovarian cancer. According to certain embodiment, the alternatively spliced mRNA isoform is TIF-IA mRNA. According to another certain embodiment, the disease is lymphoma.

According to yet another aspect, the present invention provides a method for preventing or inhibiting a disease in a subject in need thereof, comprising treating the subject in need thereof with a therapeutically effective amount of a pharmaceutical composition containing as an active ingredient an oligonucleotide capable of silencing the polynucleotide of the invention or fragments thereof.

According to one embodiment, the silencing polynucleotide is selected from the group consisting of: antisense nucleotide sequence, sense nucleotide sequence, short interfering RNA, ribozyme and aptamer.

Other objects, features and advantages of the present invention will become clear from the following description and drawings.

### BRIEF DESCRIPTION OF THE DRAWINGS

FIG. 1 demonstrates a model of exonization.

FIG. 2 presents positions 290-271 in the Alu sequence, according to the numbering in Jurka et al. (J. Mol. Evol., 32:105, 1991).

FIG. 3 exhibits a set of mutations within the 3′SS in ADAR2 gene (A) and the corresponding splicing products (B) wherein Lane 1 is a DNA size marker; lane 2, is vector only (pEGFP); lane 3 the splicing products of wt ADAR2 and lanes 4-16 are the splicing products of mutated ADAR2 corresponding to (A).

FIG. 4 presents the effect of hSlu7 on AG selection in ADAR2 3′SS sequence of and three insertion mutants thereof (A; potential AGs are in bold, and selected AG is bold and underlined) and their splicing products (B; Lanes 4-6 represent co-transfection of the insertion mutants with a plasmid expressing hSlu7; lanes 7-9 represent the insertion mutants without additional hSlu7).

FIG. 5 is the products (B) of splicing assays in wild-type and mutated putative glucosyltransferase (PGT) shown in (A).

FIG. 6 shows selected Alu-derived exons comprising 5′ss aligned for the region near the most prevalent 5′ss on the right arm of these exonized Alu sequences (in the antisense orientation).

FIG. 7 presents a comparison of exonized Alus to non-exonized intronic Alus: (A) Profile of 166,276 full-length antisense intronic Alus; (B) Per-position comparison between the 25 exonized Alus (“observed”) and non-exonized intronic antisense Alus; (C) Profile as in panel (A) but for 136,151 full-length sense intronic Alus; (D) Per-position comparison between the 136,151 full-length sense intronic Alus and 25 exonized Alus.

FIG. 8 exhibits splicing assays on ADAR2 mini-gene mutants using compensatory U1 mutants: (A) Analysis of GC introns—top line is mutants in the U1 gene and bottom line is mutants in the ADAR2 5′ss sequence, numbered according to panel C; (B) Analysis of GT introns. (C). Schematic illustration of the base paring between the 5′ss of exon/intron 8 of ADAR2 and U1. ADAR2 and U1 positions are numbered forward and reverse, respectively. Watson-Crick and non Watson-Crick base-pairing are marked by solid or dashed line, respectively.

FIG. 9 presents the strengths of 5′ss: (A) Shapiro-Senaphthy score for 5′ss strength (Shapiro et al., Nucleic Acids Res. 15:7155, 1987), calculated for the ADAR2 5′ss mutants indicated in FIG. 8A-B; (B) Free energy (ΔG) for 5′ss:U1 binding in ADAR2 5′ss mutants; (C) Shapiro-Senaphthy score for the 25 exonized Alus indicated in FIG. 6; (D) Free energy (ΔG) for 5′ss:U1 binding in 5′ss of the 25 exonized Alus.

FIG. 10 shows a potential ESE site in exon 8 of ADAR2 which is not involved in alternative splicing regulation: (A) The ADAR2 sequence from position −40 to −60 upstream of the 5′ss whereas SR protein potential sites are marked with a solid line, broken line indicates potential site that was enhanced by the mutation in position −51 from T to A and the type and binding score of each site is indicated. (B) Transfection or co-transfection in 293T cells wherein lane 1 is a DNA size marker, lane 2 is vector only (pEGFP-C3), lane 3 consists of splicing products of WT ADAR2, lane 4 consists of splicing products of the T to A mutant in position −51 that enhanced the ESE of SC35—I from a score of 2.4 to 4.2 (−51 T>A) and lanes 5 to 9 are the splicing products of WT ADAR2 with the indicated SR/A1 hnRNP protein.

FIG. 11 exhibits the sequence elements needed for the creation of an alternatively spliced Alu exon.

FIG. 12 shows the presence of exonized intronic Alu in ovarian cancer. (A) A BLAT alignment of the human ACAD-9 gene (Zhang et al., ibid) with Expressed Sequence Tags (ESTs) and mRNA using UCSC genome browser. (B) RT-PCR products of ACAD-9 alternatively spliced isoforms, regular (220 bp) without the Alu exon and an additional splicing products containing the Alu exon (310 bp), in 19 different cytoplasmic mRNA of ovarian cancer separated on 2% agarose gel. (C) similar analysis to panel B on the total cytoplasmic mRNA of the following human cell lines: 293T—adenocarcinome kidney; DU-145—prostate carcinoma; Hep62—hepatocarcinoma; PC3—prostate adenocarcinoma; MCF-7—breast adenocarcinoma; HT1080—fibrosarcoma; BM—human mesenchymal stem-cells (bone marrow); 393—lymphoblast; ES-2—clear cell carcinoma of ovarian fibroblast; SKOU3—epithelial ovarian adenocarcinoma.

FIG. 13 presents exonization of an intronic Alu in TIF-IA: (A) A BLAT alignment of the human TIF-IA gene with the ESTs and mRNA using UCSC genome browser; (B) A schematic presentation of the exonized Alu derived from Alu element of the Sx subfamily.

FIG. 14 exhibits mRNA isoforms of TIF-IA gene in various lymphoid cells.

FIG. 15 is a schematic presentation of the putative binding site, for binding SRP9/14 proteins, in Alu exons contained within TIF-IA gene.

### DETAILED DESCRIPTION OF THE PREFERRED EMBODIMENTS

The present invention provides novel genetic elements that are hitherto uncharacterized markers of cancer, genetic diseases or predisposition to genetic disease. It is now disclosed that these novel markers comprise exons derived from Alu elements and that these novel markers are useful for prognosis, diagnosis or treatment of genetic diseases.

According to one aspect the present invention provides an isolated polynucleotide comprising at least one Alu-derived exon comprising a sequence derived by alternative splicing of an Alu element, the Alu element comprising at least one mutated splice site. According to particular embodiments the mutated splice site is as set forth in any one of SEQ ID NOS:1-58.

The terms “Alu element” or “Alu sequence” are interchangeably used herein to describe short, interspersed (with an average spacing of 4 Kb), repetitive elements (SINEs) about 300 nucleotides in length, which amplify in primate genomes through a process of retroposition. These elements have reached a copy number of about 1.4 million in the human genome, comprising more than 10% of it. A typical Alu is a dimer, built of two similar sequence elements (left and right arms) that are separated by a short A-rich linker. Most Alus have a long poly-A tail of about 20-100 bases.

According to one embodiment, the mutated splice site of the Alu element is present in a sequence having at least 80% homology, preferably 85%, more preferably 90%, most preferably 95% or more homology to any one of SEQ ID NOS:1-58.

The term “X % homology” is not intended to be limited to sequences having an X % homology over the entire length of the polynucleotide but is intended to include X % homology occurring in identified functional areas therein, preferably, in areas comprising splice site involved in Alu exonization.

Without wishing to be bound by any particular theory or mechanism of action, Alu introns transform into Alu exons, also termed herein Alu-derived sequences, upon evolution of certain mutations within pseudo splice-sites (SS) in the intronic Alu sequences, a process referred herein as exonization.

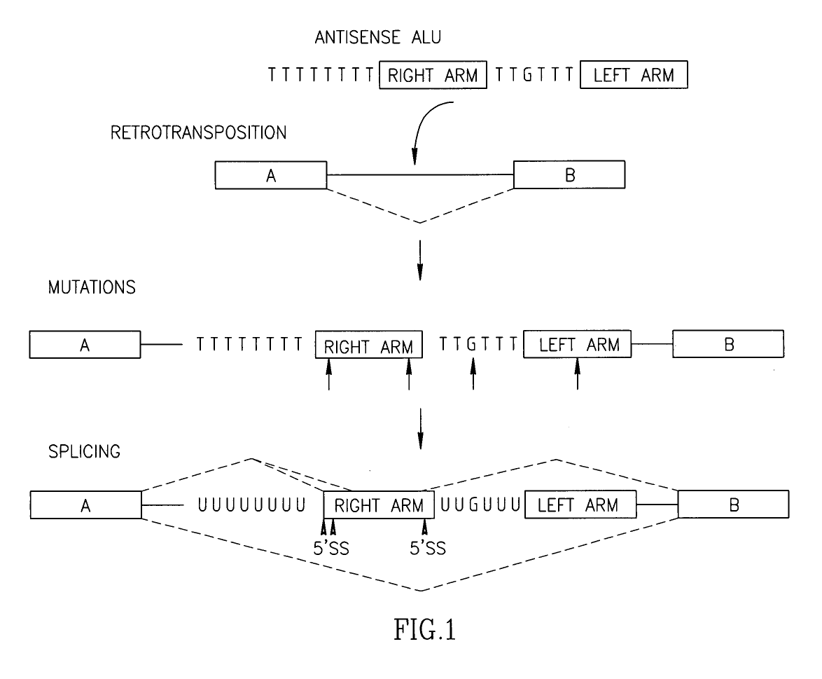

The term “exonization” as used herein is to be construed in its most general sense and refers to insertion of genetic material, preferably introns, more preferably polynucleotides derived from Alu gene, into mature mRNAs by way of splicing (FIG. 1). According to one embodiment, parts of Alu elements, predominantly on their antisense orientation, undergo exonization. Without wishing to be bound by any particular theory or mechanism of action the exonization of intronic Alu sequences is facilitated by sequence motifs that resemble splice-sites, which are found within the Alu sequence (Sorek et al. 2002, ibid).

Alternative splicing is a major mechanism that diversifies the genetic information by producing more than one mRNA from a single gene. Bioinformatics studies showed that 32-59% of human genes are involved in alternative splicing. Alternative splicing is often regulated either according to cell type, developmental stage, and sex, or in response to an external stimulus. Aberrant regulation of alternative splicing has been implicated in an increasing number of human diseases, including cancer The availability of the complete human genome sequence has made it clear that gene number is not the sole determinant of proteome complexity. Sequencing of the human genome showed that humans possess >30,000 protein coding genes, which is only 1.5-fold the number of genes in D. melanogaster. This surprisingly low number contrasts with the number of human proteins, estimated to be more than 90,000. Clearly, alternative splicing contributes to this huge number of proteins that are generated from the low number of genes.

The inventors of the present invention discovered particular positions within Alu sequences that are most commonly used as splice-sites in Alu exonization. Based on these findings the inventors of the present invention identified 7,810 Alu sequences (Sorek et al. 2004, ibid).

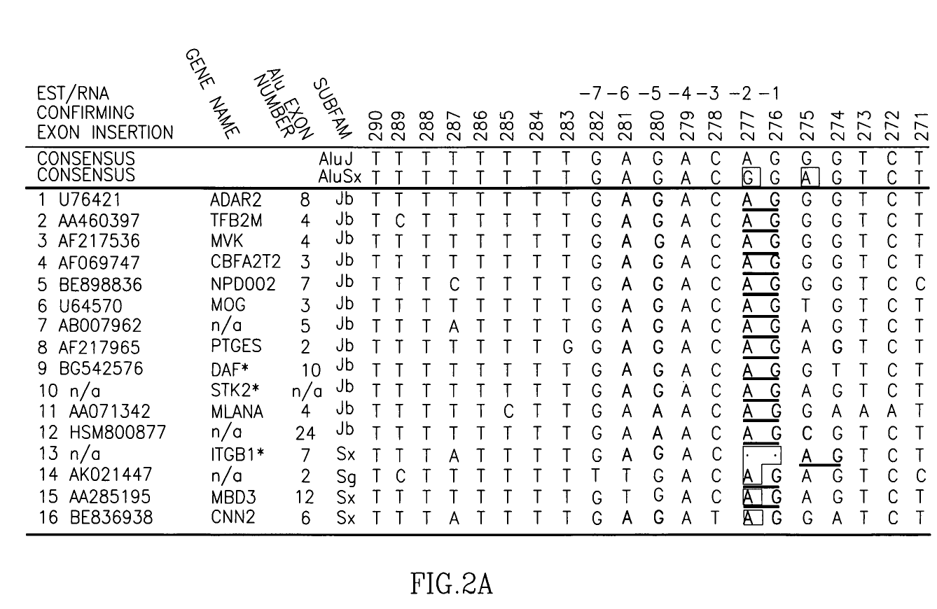

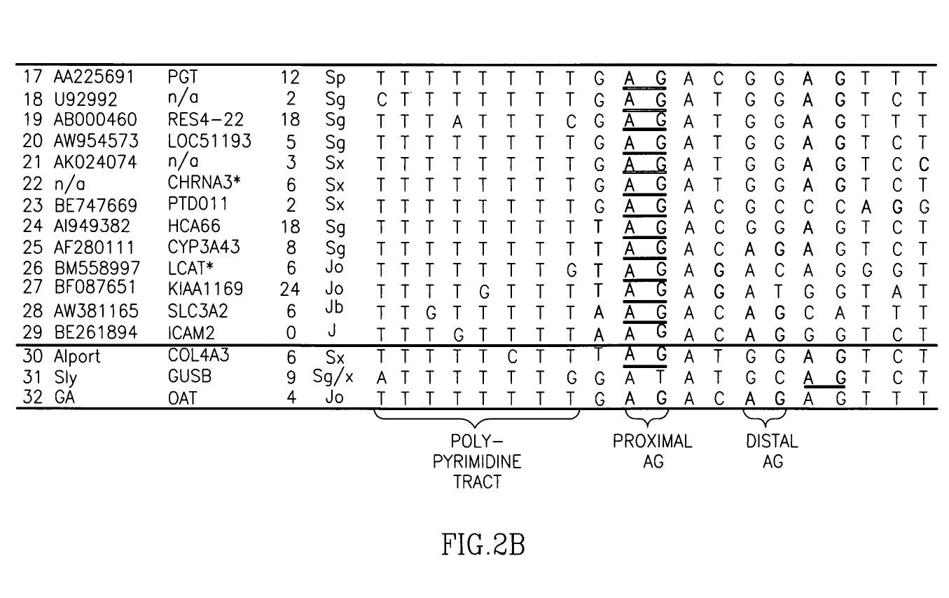

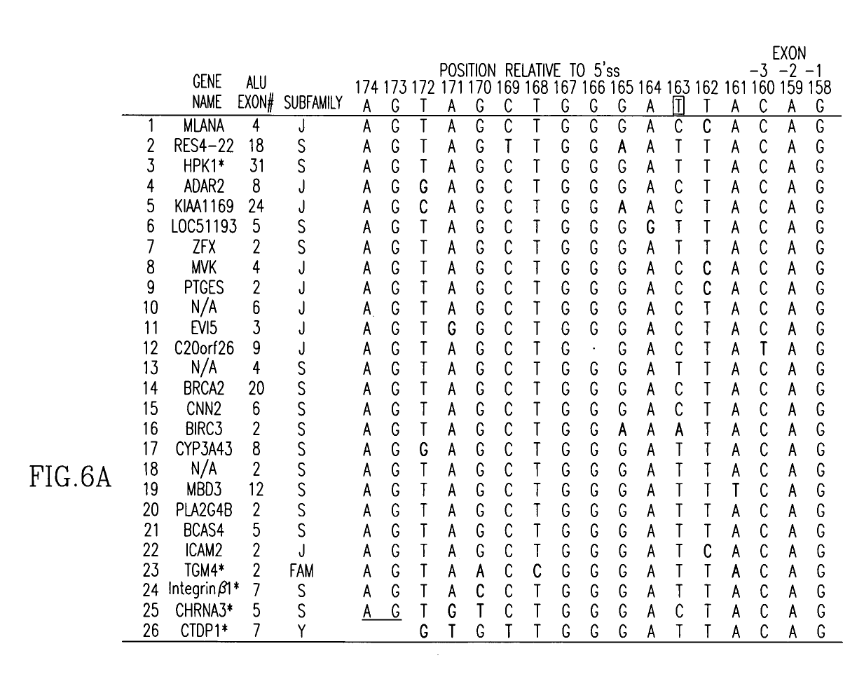

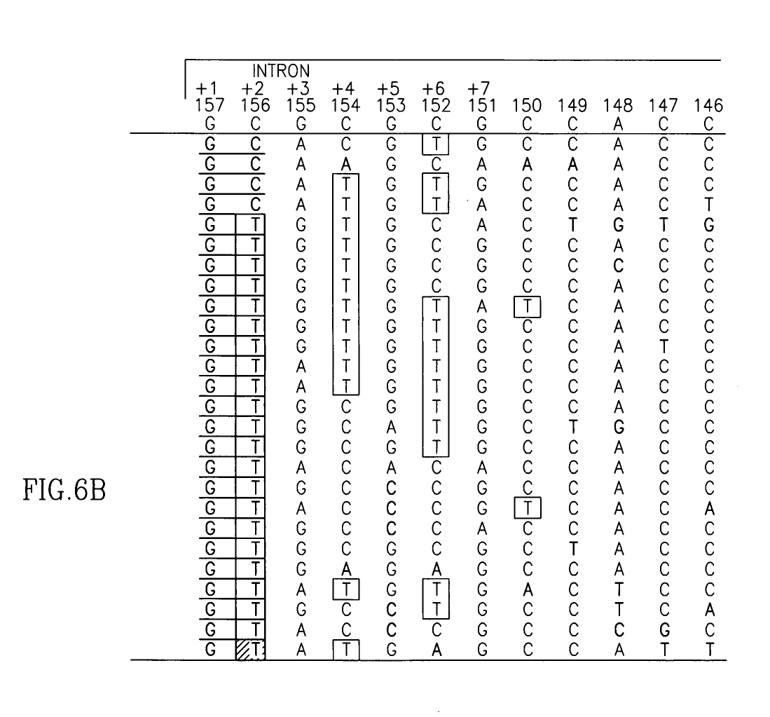

Thus, according to another embodiment, the polynucleotides of the present invention comprise alternatively spliced mRNAs that result from mutated splice-sites. According to some embodiments, the mutated splice-sites are 3′ splice-site as set forth in positions 12 to 16 of SEQ ID NOS:1-32 (corresponding to positions 275 to 279 in the consensus Alu sequence; FIG. 2). According to some embodiments, the mutated splice-sites are 5′ splice-site as set forth in positions 13 to 19 of SEQ ID NOS: 33-58 (corresponding to positions 154 to 160 in the consensus Alu sequence; FIG. 6).

In referring to 5′ or 3′ for a polynucleotide sequence, the direction of transcription is with 5′ being upstream from 3′. Thus, sequences downstream to the 3′ splice-site within an Alu element, as set forth in positions 12 to 16 of SEQ ID NOS:1-32, correspond to the 5′ end of the exonized Alu and sequences upstream to the 5′ splice-site as set forth in positions 13 to 19 of SEQ ID NOS: 33-58 correspond to the 3′ end of the exonized Alu.

According to another aspect the present invention provides an oligonucleotide comprising at least a portion of an Alu-derived exon selected from an oligonucleotide probe and an oligonucleotide primer. According to one embodiment, said oligonucleotide further comprises a detectable label. The labels include, but are not limited to, fluorophores, chromophores, radioactive isotopes, electron dense reagents, enzymes, enzymatic substrates and ligands.

It is to be understood explicitly that the scope of the present invention encompasses homologs, analogs, variants and derivatives, including but not limited to shorter and longer oligonucleotides or polynucleotides, with one or more nucleic acid substitution, as well as nucleic acid derivatives, non-natural nucleic acids and synthetic nucleic acids as are known in the art, with the stipulation that the homologs, analogs, variants and derivatives must preserve the hybridization properties of the original molecule.

It is now disclosed for the first time that out of the at least 238,000 antisense Alu sequences located within introns in the human genome 52,935 Alus carry a potential ADAR2-like (Slavov et al., ibid) 3′SS and 23,012 carry a potential PGT-like (NCBI GenBank Access No.: AA225691) 3′SS suggesting that many of these silent intronic Alu elements might be susceptible to exonization by the same single point mutation, and are thus under strict selective pressure. Such point mutations in human genomic antisense Alu sequences may be the molecular basis for predisposition to previously uncharacterized genetic diseases.

The terms “polynucleotide” refers to a polymer of nucleotides comprising a series of nucleic acids in a 5′ to 3′ phosphate diester linkage. The polynucleotide may be a single or double stranded DNA sequence. If the polynucleotide of the present invention is a single stranded DNA, then its complementary nucleic acid sequence is also within the scope of the present invention. The term “oligonucleotide” as used herein refers to a short polynucleotide, comprising about 200 nucleotides, preferably about 100, more preferably about 50 nucleotides or less.

According to another embodiment, the polynucleotide of the invention is recombinant. As used herein, a “recombinant” nucleic acid or protein molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

The term “genetic disease” as used herein refers preferably to diseases derived from somatic mutations. Somatic mutations are mutations occurring only in certain tissues, e.g., in the tumor tissue, and are not inherited in the germline. Germline mutations can be found in any tissues or cells and are inherited. Somatic gene mutations result when there is an alteration of the nucleotide sequence of a gene encoding sequence. Such change may include a substitution of one nucleotide for another, or the insertion or deletion of one or more nucleotides, resulting in a change in the corresponding amino acid sequence of which is encoded. Somatic gene mutations are distributed largely stochastically in that, with some exceptions, they are distributed at random among the genes of the genome. However, mutations are more likely to occur in cells which are undergoing mitosis, or cell division, because they often result from inaccurate DNA replication. Thus, a cell lineage which has undergone repeated divisions, as is the case in clonal amplification, has a higher probability of accumulating somatic gene mutations. Many diseases are believed to stem from somatic genetic mutation rather than inherited gene abnormalities, including different types of cancers and neurodegenerative diseases. Often, diseases, which are caused by somatic mutation, are age-related.

According to yet another embodiment, said polynucleotide further comprises a detectable label. The labels include, but are not limited to, fluorophores, chromophores, radioactive isotopes, electron dense reagents, enzymes, enzymatic substrates and ligands.

According to one embodiment, the present invention provides a vector comprising the isolated polynucleotide of the invention. According to another embodiment, the present invention provides a vector comprising the oligonucleotide of the invention. According to yet another embodiment, the present invention provides a construct comprising the oligonucleotide of the invention.

By “vector” is meant a polynucleotide molecule, preferably a DNA molecule derived, for example, from a plasmid, bacteriophage, or plant virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin™) and the hph gene which confers resistance to the antibiotic hygromycin B.

According to another aspect, the present invention provides a method for diagnosing, detecting or predicting a disease, comprising determining the presence in a biological specimen of a polynucleotide comprising a sequence selected from: a) an Alu-derived sequence comprising at least one mutated alternative splice site; and b) an alternatively spliced mRNA isoform comprising an Alu-derived exon.

According to one embodiment, the Alu-derived sequence of a) comprises a sequence as set forth in any one of SEQ ID NOS: 1-58, fragments or extensions thereof.

According to another embodiment, the disease is selected from the group consisting of: cancer, a genetic disease, a chromosomal abnormality, a genetic predisposition and a viral infection. According to another embodiment, the genetic disease is selected from the group consisting of: Alport syndrome, Sly syndrome, CCFDN (congenital cataracts, facial dysmorphism, and neuropathy) syndrome and OAT deficiency.

According to yet another embodiment, the disease is cancer selected from the group consisting of: prostate cancer, breast cancer, ovarian cancer, lung cancer, melanoma, renal cancer, bladder cancer, fibrosarcoma, hepatocellular carcinoma, osteocarcinoma, primary ductal carcinoma, giant cell sarcoma, ductal carcinoma, Hodgkin's disease, colorectal carcinoma, lymphoma, transitional cell carcinoma, uterine sarcoma, adenocarcinoma, plasmacytoma, epidermoid carcinoma, Burkitt's lymphoma, Ewing's sarcoma, gastric carcinoma, squamous cell carcinoma, neuroblastoma and rhabdomyosarcoma.

According to another embodiment, the present invention provides a method for detecting a disease, comprising
(a) obtaining a biological specimen;
(b) contacting the biological specimen with a nucleic acid probe capable of hybridizing specifically the polynucleotide of the invention or fragments thereof, under conditions suitable for hybridization; and
(c) determining the presence of a hybrid in said biological specimen.

According to another embodiment, the method further comprises:
(a) obtaining a biological specimen;
(b) generating a nucleic acid probe capable of forming a hybrid with an alternatively spliced mRNA isoform or fragments thereof;
(c) contacting the biological specimen with the nucleic acid probe under conditions suitable for hybridization; and
(d) determining the presence of the hybrid in said biological specimen.

According to yet another embodiment, the method comprises obtaining the alternatively spliced mRNA isoform by specific hybridizations, each hybridization is for of a single oligonucleotide probe complementary to a known sequence of an individual molecule. Hybridization methods include, but are not limited to, Northern blot analysis, RNase protection, in situ hybridization or selective hybridization to arrayed cDNA libraries (see Sambrook et al., Molecular cloning, A laboratory manual, Cold Spring Harbor press, NY, 1989) summarized infra.

(i) EST sequencing. The basic idea is to create cDNA libraries from tissues of interest, pick clones randomly from these libraries and then perform a single sequencing reaction from a large number of clones. Each sequencing reaction generates 300 base pairs or so of sequence that represents a unique sequence tag for a particular transcript. An EST sequencing project is technically simple to execute since it requires only a cDNA library, automated DNA sequencing capabilities and standard bioinformatics protocols. To generate meaningful amounts of data, however, high throughput template preparation, sequencing and analysis protocols must be applied. As such, the number of new genes identified as well as the statistical significance of the data is proportional to the number of clones sequenced as well as the complexity of the tissue being analyzed (Hillier et al., Genome Res. 6:807, 1996).

(ii) Subtractive cloning. Subtractive cloning offers an inexpensive and flexible alternative to EST sequencing and cDNA array hybridization. In this approach, double-stranded cDNA is created from the two-cell or tissue populations of interest, linkers are ligated to the ends of the cDNA fragments and the cDNA pools are then amplified by PCR. The cDNA pool from which unique clones are desired is designated the “tester”, and the cDNA pool that is used to subtract away shared sequences is designated the “driver”. Following initial PCR amplification, the linkers are removed from both cDNA pools and unique linkers are ligated to the tester sample. The tester is then hybridized to a vast excess of driver DNA and sequences that are unique to the tester cDNA pool are amplified by PCR. The primary limitation of subtractive methods is that they are not always comprehensive. The cDNAs identified are typically those, which differ significantly in expression level between cell-populations and subtle quantitative differences are often missed. In addition each experiment is a pair wise comparison and since subtractions are based on a series of sensitive biochemical reactions it is difficult to directly compare a series of RNA samples.

(iii) Differential display. Differential display is another PCR-based differential cloning method (Liang and Pardee, Science 257:967, 1992). In classical differential display, reverse transcription is primed with either oligo-dT or an arbitrary primer. Thereafter an arbitrary primer is used in conjunction with the reverse transcription primer to amplify cDNA fragments and the cDNA fragments are separated on a polyacrylamide gel. Differences in gene expression are visualized by the presence or absence of bands on the gel and quantitative differences in gene expression are identified by differences in the intensity of bands. Adaptation of differential display methods for fluorescent DNA sequencing machines has enhanced the ability to quantify differences in gene expression (Kato, Nucleic Acids Res. 18:3685, 1995).

A limitation of the classical differential display approach is that false positive results are often generated during PCR or in the process of cloning the differentially expressed PCR products. Although a variety of methods have been developed to discriminate true from false positives, these typically rely on the availability of relatively large amounts of RNA.

(iv) Serial analysis of gene expression (SAGE). this DNA sequence based method is essentially an accelerated version of EST sequencing (Valculescu et al., Science 270:484, 1995). In this method a digestible unique sequence tag of 13 or more bases is generated for each transcript in the cell or tissue of interest, thereby generating a SAGE library. Sequencing each SAGE library creates transcript profiles. Since each sequencing reaction yields information for twenty or more genes, it is possible to generate data points for tens of thousands of transcripts in modest sequencing efforts. The relative abundance of each gene is determined by counting or clustering sequence tags. The advantages of SAGE over many other methods include the high throughput that can be achieved and the ability to accumulate and compare SAGE tag data from a variety of samples, however the technical difficulties concerning the generation of good SAGE libraries and data analysis are significant.

Since a single human cell is estimated to express 10,000-30,000 genes, single probe methods to identify all sequences in a complex sample are ineffective and laborious. Thus, according to yet another embodiment, the method comprises obtaining the alternatively spliced mRNA isform in silico from a library of sequences selected from the group consisting of: cDNA, EST and mRNA.

The term “expressed sequence tags”, or “EST”, refers to sequences of randomly picked cDNA clones that were sequenced in a large-scale single-pass to generate over 500,000 human cDNA clones, ESTs, as described in Genome Res., 6:807, 1996. ESTs provide a large reservoir of information on human genes and are potentially powerful tools for discovery of disease genes and regional gene mapping to individual chromosomes particularly since less than 5% of the approximately 100,000 genes in the human genome have been sequenced and assigned biological functions. A number of ESTs from genes expressed in normal, precancerous, and cancerous tissues have been generated, and distributed according to various classifications, in a large number of EST libraries. In an attempt to provide more accurate genetic information, numerous methods have been developed for constructing full-length cDNA libraries from different tissue samples. For example, U.S. Pat. No. 6,265,165 provides a method for cloning of EST-specific full-length cDNA and/or full-length cDNA library. The method involves cDNA synthesis from highly enriched, homogeneously purified mRNAs and includes hybrid selection for purification of specific mRNAs from total RNA by employing antisense oligonucleotide primers of EST sequences in a single or multiplex approach. Devices and computer systems have been developed for collecting information about gene expression or expressed sequence tag (EST) expression in large numbers of tissue samples. For example, PCT application WO92/10588, incorporated herein by reference for all purposes, describes techniques for sequencing or sequence checking nucleic acids and other materials. Probes for performing these operations may be formed in arrays according to the methods of, for example, the pioneering techniques disclosed in U.S. Pat. No. 5,143,854; U.S. Pat. No. 5,571,639 and U.S. Pat. No. 6,308,170 all three incorporated herein by reference for all purposes.

According to a preferred embodiment, the alternatively spliced mRNA isoform is an ACAD-9 mRNA (Zhang et al., ibid). According another preferred embodiment, the disease is ovarian cancer. According to certain embodiment, the alternatively spliced mRNA isoform is TIF-IA mRNA. According to another certain embodiment, the disease is lymphoma.

According to yet another embodiment, the biological specimen is derived from a human or a non-human primate. According to yet another embodiment, the biological specimen is selected from a tissue sample, fluids, cells, cellular extract, tissue extract and genetic material extracted from same, including but not limited to DNA and RNA. According to yet another embodiment, the fluids are selected from the group consisting of: blood, urine, sperm and saliva. According to certain embodiments the biological specimen is fixed tissue microsections selected from: frozen section and paraffin sections among others. The method for detecting a disease in the microsection may be in-situ hybridization (ISH) as known in the art.

According to yet another embodiment, the nucleic acid probe is labeled. The labels include, but are not limited to, fluorophores, chromophores, radioactive isotopes, electron dense reagents, enzymes, and ligands having specific binding partners (e.g. biotin and avidin).

There are essentially four types of nucleic acid probes that can be used for hybridization according to the method of the present invention, wherein hybridization can be performed using in situ hybridization (ISH) protocols among others. The first type of probe is oligonucleotide probe. The oligonucleotide probes are typically produced synthetically by an automated chemical synthesis utilizing readily available deoxynucleotides. Designing the sequence of the probe is one of the more critical decisions required when using oligonucleotide probes and is not a matter of picking any region within the coding region of the target gene to bind to but requires careful design. These probes have the advantages of being resistant to RNases and are small, generally around 40-50 base-pairs. This is ideal for ISH because their small size allows for easy penetration into the cells or tissue of interest. In addition, because they are synthetically designed, it is possible to make a series of probes that have the same GC content; Since G/C base pairs bond more strongly than A/U base pairs, differences in GC content would require different hybridization conditions, so with oligonucleotides protocols can be standardized for many different probes irrespective of the target genes being measured. Another advantage of the oligonucleotide probes is that they are single stranded therefore excluding the possibility of renaturation. Benefits of using oligonucleotide probes.

The other type of probe that may be utilized for hybridization in accordance with the principles of the present invention is single stranded DNA probe. The single stranded DNA probes have similar advantages to the oligonucleotide probes except they are much larger, probably in the 200-500 bp size range. They can be produced by reverse transcription of RNA or by amplified primer extension of a PCR-generated fragment in the presence of a single antisense primer. That is, once the sequence of interest is amplified, a subsequent round of PCR is carried out using the first PCR product as template, but only using the anti-sense primers, thus producing single stranded DNA. This is therefore their disadvantage.

The third type of probe that may be utilized for hybridization in accordance with the principles of the present invention is the double stranded DNA probe. It can be produced by the inclusion of the sequence of interest in a bacteria which is replicated, lysed and the DNA extracted, purified and the sequence of interest is excised with restriction enzymes. On the other hand, if the sequence is known then by designing appropriate primers one can produce the relevant sequence very rapidly by PCR, potentially obtaining a very clean sample. The advantage of the bacterial preparation is that it is possible to obtain large quantities of the probe sequence in question. Because the probe is double stranded, it means that denaturation or melting has to be carried out prior to hybridization in order for one strand to hybridize with the mRNA of interest. These probes are generally less sensitive because of the tendency of the DNA strands to rehybridize to each other and are not as widely used today.

A forth type of probe that may be utilized for hybridization in accordance with the principles of the present invention is RNA probe (cRNA probe or riboprobe). RNA probes have the advantage that RNA-RNA hybrids are very thermostable and are resistant to digestion by RNases. This allows the possibility of post-hybridization digestion with RNase to remove non-hybridized RNA and therefore reduces the possibility of background staining. There are two methods of preparing RNA probes: (a) complimentary RNAs are prepared by an RNA polymerase-catalyzed transcription of mRNA in the 3′ to 5′ prime direction; and (b) in vitro transcription of linearized plasmid DNA with RNA polymerase can be used to produce the RNA probes. Here plasmid vectors containing polymerase from bacteriophages T3, T7 or SP6 are used.

These probes however can be difficult to work with as they are very sensitive to RNases (ubiquitous RNA degrading enzymes) and so, scrupulous sterile technique should be observed or these probes can easily be destroyed. RNA probes may still be used according to the teaching present invention, with in situ hybridization.

Oligonucleotide gene probes have multiple advantages over RNA or cDNA probes when used for in situ hybridization in the context of the present invention. The major advantages are as follows: stability (in particular, they are not degraded by RNAses), availability, faster and less expensive to use, easier to work with, more specific, better tissue penetration, better reproducibility and a wide range of labeling methods that do not interfere with target detection.

For detecting the desired oligonucleotides in frozen tissue sections, using ISH, the following probes and detection means are typically used:
1. ³⁵S-labeled probe, Detection by emulsion or film autoradiography
2. GreenStar* FITC-labeled probe, Detection by direct fluorescence, AP (alkaline phosphatase) or tyramide signal amplification (TSA).
3. GreenStar* DIG-labeled probe, detection by direct fluorescence, AP (alkaline phosphatase) or TSA.
4. GreenStar* BIOTIN-labeled probe, detection by AP or HRP (Horseradish Peroxidase) with or without TSA.

For detecting the desired oligonucleotides in paraffin tissue sections, using ISH, the following probes and detection means are typically used:
1. GreenStar* BIOTIN-labeled probe, Detection by AP (alkaline phosphatase) or HRP with or without TSA.
2. GreenStar* DIG-labeled probe, Detection by direct fluorescence, AP or TSA.
3. DIG-labeled probe, detection by AP, FITC or rhodamine.

There are several options for labeling a nucleic acid probe, which are applicable for use in the present invention. Classically oligonucleotide probes are either 5′ or 3′ end-labeled or 3′ tailed with modified nucleotides that have a “label” attached that can be detected after the probe has hybridized to its target. With end-labeling a single modified ddNTP (that incorporates the label) is added to either the 5′ or the 3′ end of the molecule enzymatically or during probe synthesis. 3′ tailing involves addition of a tail (on average 5-50 nucleotides long of modified dNTPs depending on the method used) using the enzyme terminal transferase (TdT). A recently developed non-enzymatic proprietary labeling technology, GreenGene™ allows for covalent 3′ labeling of oligonucleotides subsequent to probe synthesis. Using this technology an optimized number of labels (either Biotin, FITC, rhodamine or DIG) separated by proprietary linker molecules can be added to the 3′ end of an oligonucleotide probe in one chemical step. Radiolabeled probes, such as ³⁵Sulphur (³⁵S) radioisotope, may be used in the present invention. The radiolabeled probes may be then visualized by exposure of the tissue section or cells (to which the labeled oligonucleotide has be hybridized) against photographic film which is then developed. Instead of using photographic film to detect the probe within the tissue section, the slide containing the section of interest may also be dipped into a photographic emulsion which is allowed to dry. The slide is stored in the dark at −80° C. to allow the slide emulsion to become exposed. After the incubation period, the slides are then developed in the same way as normal photographic film wherein the black silver grains indicate the sites of the labeled transcripts. This is particularly useful for investigating gene expression on a cell by cell level. If immunocytochemistry is performed on the tissue before in situ hybridization, it is possible to examine gene expression in phenotypically defined cell populations at single cell resolution.

On the other hand several non-radioactive labels may be used in the present invention, include Biotin, digoxin and digoxigenin (DIG), alkaline phosphatase and the fluorescent labels, fluorescein (FITC), Texas Red and rhodamine.

Labeling the probe may be achieved using suitable kits, such as GreenStar™ labeled probes. Probes may be 5′ end labeled (addition of a biotin-, rhodamine-, FITC-modified nucleotide to the 5′ end) during the synthesis cycle if required. For 3′ tailing both radiolabels and non-radioactive labels are “attached” to the single stranded oligonucleotide probe by using the enzyme terminal transferase to add a tail of labeled dioxy nucleotide(s) (dNTPs, for example ³⁵S-dATP, DIG-dUTP, Biotin-dUTP or FITC-dUTP) to the 3′ end of the oligonucleotide. Many commercial kits are available that allow to generate 3′ tailed probe (e.g. DIG tailing kits of Roche Molecular Biochemical). It is also possible to end label the 3′ end of the oligonucleotide probe if modified di-deoxy nucleotides (ddNTPs) are used as the substrate for TdT, for example by using DIG-ddUTP instead of DIG-dUTP with a slightly modified protocol. In this case TdT will add a single modified and labeled nucleotide to the 3′ end of the oligonucleotide facilitating later detection.

In contrast to adding the labeled nucleotides to either end of the oligonucleotide probe it is also possible to have labels incorporated into the oligonucleotide when it is being synthesized, for example by adding biotin- or FITC-labeled dATP in place of non-labeled dATP during synthesis so that a label or “tag” appears every time that the ATP nucleotide appears in the probe sequence. However with this method there is the strong possibility especially with short oligonucleotide probes of causing significant disruption of the hybridization of the probe to the target sequence in the tissue.

As mentioned, radiolabeled probes are detectable using either photographic film or photographic emulsion. The fluorescent labels described above are detectible “directly” by using a fluorescent microscope or plate reader to examine the tissue or cells on which the labeled oligonucleotide probe has hybridized to. The use of fluorescent labels with in situ hybridization has come to be known as FISH (fluorescent in situ hybridization) and one advantage of these fluorescent labels is that two or more different probes can be visualized at one time. Additionally FITC labeled probes can be detected using anti-FITC antibodies available from many scientific supply houses.

In contrast both Biotin and DIG labeled oligonucleotide probes generally require an intermediate step(s) before detection of the probe can occur and they are thus detected “indirectly” much like in a typical immunocytochemistry protocol where it would be unusual to have the label on the primary antibody, rather a secondary labeled antibody is used to detect the primary antibody in the tissue section. Specific anti-DIG antibodies can be used to detect the presence of a DIG-labeled probe. Digoxigenin (DIG) is a steroid isolated from the digitalis plant and as the blossoms and leaves are the only known source of digoxigenin, the anti-DIG antibodies are not likely to bind to other biological material. The digoxigenin is linked by a spacer arm containing 11 carbon atoms to the C-5 position of the uridine nucleotide. The advantage of using a DIG labeled probe is that it can be detected with antibodies conjugated to a number of different labels such as alkaline phosphatase, which results in a blue precipitate when the enzyme is incubated in the presence of the substrate NBT/BCIP (Tetrazoliun salt/5-bromo-4-chloro-3 idolyl-phosphate) or becomes a fluorescent label when incubated with HNPP (2-hydroxy-3-naphthoic acid-2′-phenylanilide phosphate). The Anti-DIG antibodies can be conjugated to other labels that require no development, such as FITC, Texas Red or Rhodamine. In some protocols anti-DIG antibodies may be unlabelled wherein a secondary conjugated antibody is used to visualize the probe. Being closely related in structure to digoxigenin, GreenStar™ digoxin hyperlabeled probes can be detected using the same methods and kits designed for digoxigenin detection.

Biotin is the other common compound used in the labeling of oligonucleotide probes. Linked to ATP (other nucleotides have also been biotinylated) it can be detected with antibodies but more often a 65 kd glygoprotein Avidin from egg white or Streptavidin from the fungi Streptomyces avidinii is used, as they have a high binding capacity to biotin and can be conjugated to a similar range of visual and fluorescent labels.

In general, of the two “indirect” labels it is thought that the DIG label is more sensitive than the Biotin label and that the DIG label allows comparable sensitivity to ³⁵S radiolabeled probes.

Applying in situ hybridization for carrying out the present invention, apart from the necessary controls the most important issue is permeablization of the sectioned tissue in order to enable the probe to reach the target, that is the mRNA of the target gene located within the fixed tissue section or the fixed sample of cells. The act of fixation results in cross-linking of proteins, which may present an obstacle to good infiltration of the probe, and finally mRNA sequences are often surrounded by proteins which may mask the target sequence. Therefore permeabilization is essential. There are a number of different elements in permeabilization procedures, wherein the following three reagents are commonly used to permeabilize tissue: HCl (typically, incubation in 0.2M HCl for 20-30 min), detergents (Triton or SDS) and Proteinase K. Detergent treatment, usually with Triton X-100 or SDS, is frequently used to permeabilize the membranes by extracting the lipids. This is not usually required in tissue that has been embedded in wax, but for intact cells or cryostat sections these may be more critical steps. Proteinase K is an endopeptidase which is non-specific and attacks all peptide bonds, is active over wide pH range and not easily inactivated. It is used to remove protein that surrounds the target sequence. Optimal concentration have to be determined but a normal starting concentration is 1 μg/ml. Incubation has to be carefully monitored because if the digestion proceeds to far you could end up destroying most of the tissue or cell integrity.

Pretreatment/Prehybridization is generally carried out to reduce background staining. Many of the non-radioactive oligonucleotide probe detection methods utilize enzymes such as peroxidases or alkaline phosphatases to visualize the label. Therefore any endogenous tissue enzymes which could result in giving a very high background should be neutralized. This can be achieved with peroxidases by treating the tissues with 1% H₂O₂ in methanol for 30 minutes. For Alkaline phosphatases, the drug levamisole may be added to the substrate solution. In general, however, this is considered to be unnecessary since residual alkaline phosphatase activity is usually lost during hybridization.

Another commonly observed pre treatment when using RNA probes is acetylation with acetic anhydride (0.25%) in triethanolamine. This treatment is thought to be important for decreasing background but it also appears to inactivate RNases and may help in producing a strong signal. Luckily RNases are not an issue when oligonucleotide probes are used. Fixation of tissue effectively “secures the target RNA” i.e. the mRNA within the tissue from RNAse digestion and oligonucleotide probes are naturally resistant to RNases. This is why solutions do not need to be RNase free when using oligonucleotide probes with in situ hybridization.

Prehybridization involves incubating the tissue/section with a solution that is composed of all the elements of the hybridization solution, minus the probe although not all protocols use a prehybridization step.

The composition of the hybridization solution is critical in controlling the efficiency of the hybridization process. Hybridization depends on the ability of the oligonucleotide to anneal to a complementary mRNA strand just below its melting point (Tm; the value of the Tm is the temperature at which half of the oligonucleotide is present in a single stranded form).

The major factors that influence the hybridization of the oligonucleotide probe to the target mRNA are: Temperature; pH; monovalent cation concentration and presence of organic solvents. Typical hybridization solutions with a hybridization temperature of around 37° C. and an overnight incubation period are: dextran sulphate; the organic solvents formamide and DTT (dithiothreitol); SSC (NaCl+Sodium citrate); and EDTA. Other components are added to decrease the chance of nonspecific binding of the oligonucleotide probe, for example: ssDNA, tRNA (acts as a carrier RNA), polyA and Denhardts solution.

Following hybridization the material is washed to remove unbound probe or probe which has loosely bound to imperfectly matched sequences. Washing should be carried out at or close to the stringency condition at which the hybridization takes place with a final low stringency wash.

The terms “stringent” or “stringency condition” as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20-25° C. below the melting temperature). One or more factors be may be varied to generate conditions of either low or high stringency.

Of course an important part of any experimental procedure is the inclusion of controls. Thus, according to one embodiment, the method further comprises contacting said biological specimen with a control nucleic acid probe capable of forming a hybrid with at least one control gene or fragments thereof. According to yet another embodiment, the at least one control gene is selected from the group consisting of: β-actin, glycer-aldehyde-phophate-dehydrogenase (GAPDH), S16 rRNA, phosphoribosyl transferase (HPRT), dihydrofolate reductase (DHFR), adenosine deaminase, phosphoglycerol kinase (PGK), pyruvate kinase, phosphoglycerol mutase.

According to yet another embodiment, the method further comprises contacting the nucleic acid probe with a biological standard. According to certain embodiments, the biological standard is derived from ostensibly healthy tissue, without any discernible pathology, of a human or a non-human primate and said biological specimen is derived from a non-healthy tissue derived from the human or a non-human primate. Alternatively, said biological specimen is derived from a non-healthy tissue derived from a different human or non-human primate.

In carrying out an in situ hybridization experiment the following controls are commonly used: polyd(T) probe (detects total mRNA poly A tails), nonsense probe (GeneDetect™), sense and antisense probes, pan-species actin probe and probes against house keeping sequences including, but not limited to, Actin and beta-tubulin.

In order to determine that the probe is only binding to the desired mRNA it is recommended to digest the tissue with RNase, in representative control samples, prior to hybridization with the oligonucleotide probe. The absence of binding after RNase treatment indicates that binding was indeed to RNA within the tissue.

For verifying specific versus non-specific binding the first control involves hybridization of the tissue with both labeled sense and antisense probes in parallel. The antisense probe in theory detects both the target mRNA and any non-specific targets it can bind to due to the chemical properties of the probe (but not due to the probe sequence). The sense control probe gives a measure of non-specific probe binding only due to the chemical properties of the probe. In essence if the sense probe detects nothing, then it may be deduced that any signal detected by the antisense probe is due to sequence-specific binding to mRNA and not due to binding to other targets within the cell. Competition studies with labeled and excess unlabeled probes also help distinguish between specific versus non-specific binding. This is because by definition specific binding is saturable (i.e. there are finite target mRNA molecules to which the probe can bind) while non-specific binding is not (there are infinite non-specific targets). Therefore excess unlabeled probe can displace (by competition for binding sites) the specific binding of the labeled probe (i.e. to the target mRNA) but not non-specific binding of the labeled probe. According to certain alternative embodiments, the tissue is prehybridized with 10× molar amount of: (1) unlabeled correct sequence oligonucleotide probe and (2) unlabelled nonsense probe before hybridizing with labeled probe. The nonsense probe should preferably have a similar CG content, a similar length and have no homology to the sequence of interest. It is important to note however that competition studies do not verify the identity of the mRNA to which the labeled probe is binding since both the labeled and unlabeled probes have the same sequence.

Another way to ensure that the probe is binding to the correct target sequence is by choosing a correct probe sequence, preferably in silico, from the start and having high stringency hybridization and wash conditions. In summary the following controls are performed in parallel with ISH experiments: poly(dT) probe hybridized to sections, RNase treatment of sections before labeled antisense probe hybridized, hybridize in parallel labeled sense and labeled antisense probes and hybridize labeled antisense probe in presence of a) 10× unlabeled antisense probe and separately in presence of b) 10×unlabeled nonsense probe.

According to yet another embodiment, the present invention provides a method for detecting a disease in a biological specimen utilizing polymerase chain reaction, the method comprising:
(a) obtaining a genetic material derived from a biological specimen;
(b) contacting the genetic material with a reaction mixture comprising a plurality of primers that are capable of amplifying the polynucleotide of the invention or fragments thereof, by polymerase chain reaction; and
(c) separating the amplified products.

According to yet another embodiment, the method comprises selecting the alternatively spliced mRNA isforms from a library of sequences selected from the group consisting of: cDNA, EST and mRNA.

According to yet another embodiment, the plurality of primers comprises at least one primer that is labeled to enable detection of the amplified products. According to some alternative embodiments, the amplified products are separated by means of molecular size exclusion and the size of the amplified products.

According to yet another embodiment, the reaction mixture comprises the four nucleoside triphosphates: dATP, dCTP, dTTP, and dGTP. According to an alternative embodiment, the reaction mixture comprises dATP, dCTP, dTTP, dGTP, and an analog of dGTP, including but not limited to, inosine, 7-deaza-guanosine, 7-deaza inosine deoxyribonucleotides and 2′-deoxy analogs thereof.

According to another embodiment, the biological specimen comprises genetic material extracted from tissue, cells and fluids. According to yet another embodiment, the fluids are selected from the group consisting of: blood, urine and saliva.

According to a certain embodiment, the biological specimen is derived from a cancer patient. According to another certain embodiment, the standard is a biological specimen derived from a healthy individual.

According to a preferred embodiment, the alternatively spliced mRNA isoform is an ACAD-9 mRNA. According another preferred embodiment, the disease is ovarian cancer. According to certain embodiment, the alternatively spliced mRNA isoform is TIF-IA mRNA. According to another certain embodiment, the disease is lymphoma.

Labels may be attached to the primer using a variety of techniques and can be attached at the 5′ end, and/or the 3′ end and/or at an internal nucleotide as mentioned above. The label can also be attached to spacer arms of various sizes, which are attached to the primer. These spacer arms are useful for obtaining a desired distance between multiple labels attached to the primer. The fluorescent dyes useful for labeling the primers are commercially available and include fluorescein dyes, rhodamine dyes, and various derivatives thereof including, but not limited to, FAM (6-carboxyfluorescein), Lucifer yellow, TAMRA (6-carboxy-tetramethyl-rhodamine) or JOE (2′,7′-dimethoxy-4′,5′-6-carboxyrhodamine). Often functional groups are introduced into the phenyl group of the dyes to serve as a linkage site to an oligonucleotide.

The PCR is performed under conditions known in the art. Typically, the range of temperatures in which denaturation of DNA is achieved is between 90° C. to 105° C. for a time period of 0.5 to 6 minutes. The annealing process during which the primer hybridizes with its template is performed at a temperature of about 37° C. to about 70° C., preferably at a temperature of about 45° C. to 70° C. for about 0.5 to 5 minutes, more preferably for 1-3 minutes. The extension of the PCR products is achieved by heating the reaction mixture to a temperature of about 40° C. to 80° C. for a period of about 0.5 to 40 minutes. The number of PCR cycles is variable but preferably the number is at least 10-20 times and at most 30-40 times.

To improve the yield of the PCR extension products, at least one additional constituent selected from DMSO, formamide, Tween 20, and tetramethylammonium chloride (TAM) are typically added to the reaction mixture at the respective concentration of 1-5%, 0.1-1%, 15-100 mM. Preferably, DMSO is added at a concentration of about 5 to 15%.

Analysis of the fluorescently labeled PCR products is performed typically on a denaturing gel. The denaturing gel may be composed of polyacrylamide and urea in a ratio of 6% polyacrylamide and 7 mM urea. Alternatively, other ratios of polyacrylamide and urea or other denaturing gels may be used as known in the art.

Monitoring the amplified product during amplification may be performed using any of a variety of real time amplification methods. For example, certain methods involve monitoring the formation of amplification products directly using labels, which bind to the amplification product to form a complex that creates a detectable signal. Alternatively, the formation of amplification products can be monitored using probes, such as fluorescently labeled probes, which are complementary to the amplification products. During the amplification process, alteration of the probe generates a detectable signal, which correlates with the formation of the amplification product. Fluorogenic nuclease assays such as the “TaqMan” assay exemplify this type of approach, wherein a probe is used to monitor amplification product formation.

According to yet another aspect, the present invention provides a method for preventing or inhibiting a disease in a subject in need thereof, comprising treating the subject in need thereof with a therapeutically effective amount of a pharmaceutical composition containing as an active ingredient a polynucleotide capable of hybridizing to an oligonucleotide comprising a sequence selected from: Alu-derived exon, alternatively spliced mRNA isoform comprising one or more Alu-derived exons and fragments thereof.

According to another embodiment, the genetic disease is selected from the group consisting of: Alport syndrome, Sly syndrome, CCFDN (congenital cataracts, facial dysmorphism, and neuropathy) syndrome and OAT deficiency.

According to one embodiment, the polynucleotide is selected from the group consisting of: antisense nucleotide sequence, sense nucleotide sequence, short interfering RNA, ribozyme and aptamer.

In recent years, advances in nucleic acid chemistry and gene transfer have inspired new approaches for treating genetic diseases by gene interference. Antisense technology has been one of the most commonly described approaches in protocols to achieve gene-specific interference. For antisense strategies, stochiometric amounts of single-stranded nucleic acid complementary to the messenger RNA for the gene of interest are introduced into the cell.

International Publication No. WO 02/10365 provides a method for gene suppression in eukaryotes by transformation with a recombinant construct containing a promoter, at least one antisense and/or sense nucleotide sequence for the gene(s) to be suppressed, wherein the nucleus-to-cytoplasm transport of the transcription products of the construct is inhibited. In one embodiment, nucleus-to-cytoplasm transport is inhibited by the absence of a normal 3′ UTR. The construct can optionally include at least one self-cleaving ribozyme. The construct can also optionally include sense and/or antisense sequences to multiple genes that are to be simultaneously downregulated using a single promoter. Also disclosed are vectors, plants, animals, seeds, gametes, and embryos containing the recombinant constructs.

European Patent Application No. 0223399 A1 describes methods for the use of genetic engineering technology in plants to achieve useful somatic changes to plants, not involving the expression of any exogenous proteins, but instead controlling the expression of an endogenous protein or other DNA or RNA factor naturally introduced into the plant cells through outside agents, such as agents of disease or infection.

Antisense has recently become accepted as therapeutic moieties in the treatment of disease states. For example, U.S. Pat. No. 5,098,890 is directed to antisense oligonucleotide therapies for certain cancerous conditions. U.S. Pat. No. 5,135,917 provides antisense oligonucleotides that inhibit human interleukin-1 receptor expression. U.S. Pat. No. 5,087,617 provides methods for treating cancer patients with antisense oligonucleotides. U.S. Pat. No. 5,166,195 provides oligonucleotide inhibitors of HIV. U.S. Pat. No. 5,004,810 provides oligomers capable of hybridizing to herpes simplex virus Vmw65 mRNA and inhibiting replication. U.S. Pat. No. 5,194,428 provides antisense oligonucleotides having antiviral activity against influenza virus. U.S. Pat. No. 4,806,463 provides antisense oligonucleotides and methods using them to inhibit HTLV-III (human T-cell lymphotropic virus type III, known also as HIV) replication. Antisense oligonucleotides have been safely administered to humans and several clinical trials of antisense oligonucleotides are presently underway. It is, thus, established that oligonucleotides can be useful therapeutic instrumentalities and that the same can be configured to be useful in treatment regimes for treatment of cells and animals, especially humans.

Aptamers are specifically binding oligonucleotides for non-oligonucleotide targets that generally bind nucleic acids. The use of single-stranded DNA as an appropriate material for generating aptamers is disclosed in U.S. Pat. No. 5,840,567. Use of DNA aptamers has several advantages over RNA including increased nuclease stability, in particular plasma nuclease stability, and ease of amplification by PCR or other methods. RNA generally is converted to DNA prior to amplification using reverse transcriptase, a process that is not equally efficient with all sequences, resulting in loss of some aptamers from a selected pool.

The methods of the invention may be further utilized for screening short interfering RNAs (siRNAs; Fire et al., Nature 391:806, 1998). RNA interference refers to the process of sequence-specific post-transcriptional gene silencing in animals mediated by siRNAs. The corresponding process in plants is commonly referred to as post-transcriptional gene silencing or RNA silencing. The process of post-transcriptional gene silencing is thought to be an evolutionarily conserved cellular defense mechanism used to prevent the expression of foreign genes and is commonly shared by diverse flora and phyla.

RNA interference is a phenomenon in which double stranded RNA (dsRNA) reduces the expression of the gene to which the dsRNA corresponds. The phenomenon of RNAi was subsequently proven to exist in many organisms and to be a naturally occurring cellular process. The RNAi pathway can be used by the organism to inhibit viral infections, transposon jumping and to regulate the expression of endogenous genes (e.g. Zamore P D., Nat Struct Biol. 8:746, 2001).

International Publication No. WO 00/01846 discloses methods for identifying specific genes responsible for conferring a particular phenotype in a cell using specific dsRNA molecules. International Publication No. WO 01/29058 discloses specific genes involved in dsRNA-mediated RNAi. International Publication No. WO 99/07409 discloses specific compositions consisting of particular dsRNA molecules combined with certain anti-viral agents. International Publication No. WO 99/53050 discloses certain methods for decreasing the phenotypic expression of a nucleic acid in plant cells using certain dsRNAs. International Publication No. WO 01/49844 discloses specific DNA constructs for use in facilitating gene silencing in targeted organisms.

International Publications Nos. WO 02/055692, WO02/055693, and EP 1144623 B1 disclose methods for inhibiting gene expression using RNAi. International Publications Nos. WO 99/49029 and WO01/70949, and AU 4037501 describe certain vector expressed siRNA molecules. U.S. Pat. No. 6,506,559, discloses methods for inhibiting gene expression in vitro using certain siRNA constructs that mediate RNAi. U.S. Pat. No. 5,681,747 discloses methods for inhibiting human-PKCα expression with an oligonucleotide specifically hybridizable to a portion of the 3′-untranslated region of PKCα.

### EXAMPLES

The following examples are to be construed in a non-limitative fashion and are intended merely to be illustrative of the principles of the invention disclosed.

Materials & Methods:

Building a Genomic Alu Profile

The Alu Gene database was used to extract full-length genomic Alu sequences that are found within introns in the antisense orientation of Alu. This search yielded 166,276 full-length Alus. Each of these Alus was aligned to its ancestral sequence using ClustalW 1.8. For each position along the Alu consensus, occurrence of each nucleotide was counted, and a frequency profile was built. In a similar manner, the sequences of 25 Alus that were exonized using position 157 as 5′ss were aligned to their consensus, and an “observed” profile was built. A parallel “expected” profile was calculated by multiplying the frequency profile matrix from the 166,276 non-exonized intronic Alus by 25 (the number of exonized sequences). Statistical significance between the observed and expected profile were calculated using Chi statistical test.

Plasmid Constructs

Oligonucleotide primers were designed to amplify (from human genomic DNA) a mini-gene that contains exons 7, 8, and 9 of the adenosine deaminase (ADAR2) gene, and exons 11, 12, and 13 of the putative glucosyltransferase (PGT) gene. Each primer contained an additional extension encoding a restriction enzyme sequence. The PCR product of ADAR2 and PGT (2.2 kb and 3 kb, respectively) was restriction digested and inserted between the KpnI/BglII sites in the pEGFP-C3 vector (Clontech). The hSlu7 cDNA was a kind gift from Robin Reed and was inserted as described above into pEGFP-C3. The U1 gene was cloned in the pCR vector.

Site-Directed Mutagenesis

Oligonucleotide primers containing the desired mutations were used to amplify the mutation-containing replica of the wild-type ADAR2 mini-gene plasmid or the U1 gene respectively. The products were treated with DpnI restriction enzyme (12U) (New England Biolabs) at 37° C. for 1 h. 1-4 μL of the mutant DNA was transformed into E.coli DH5α strain. Colonies were picked followed by mini-prep (QIAgene) and midi-prep (BRL). All plasmids were confirmed by sequencing.

Transfection, RNA Isolation and RT-PCR Amplification

293T, HeLa and HT1080 cells lines were cultured in Dulbecco's Modification of Eagle Medium, supplemented with 4.5 g/ml glucose (Biological Industries) and 10% fetal calf serum, and cultured in 60 mm dish under standard conditions at 37° C. with 5% CO₂. Cells were grown to 50% confluence, and transfection was performed using Metafectene (Biontex) with 10 μg of plasmid DNA or using FuGENE6 (Roche) with 6 μg of plasmid DNA. Cells were harvested after 48 hr. Total cytoplasmic RNA was extracted using Tri Reagent (Sigma), followed by treatment with 1U of RNase-free DNase (Promega). Reverse transcription (RT) was preformed on 2 μg total cytoplasmic RNA for 1 hr at 42° C., using a pEGFP-C1-specific reverse primer and 2U of reverse transcriptase avian myeloblastosis virus (A-AMV, Roche).

The spliced cDNA products derived from the expressed mini-genes were detected by PCR, using the pEGFP-C3-specific reverse primer and an exon 7- or 11-forward primer (ADAR2 and PGT respectively): Amplification was performed for 30 cycles, consisting of 1 min at 94° C., 45 sec at 61° C., and 1 min at 72° C. The products were resolved on 2% agarose gel and confirmed by sequencing. The level of mRNA of the house-keeping gene, Glycerol-3-phosphate dehydrogenase, was used as the internal control for each transfection.

Nonsense-Mediated Decay (NMD)

The level of the isoforms are unaffected by the Nonsense-Mediated Decay. This was indicted by incubation of the transfected cells with 300 μg/ml puromycin (Sigma) for 4 hr before RNA collection.

Real-Time PCR

The LightCycler PCR and detection system (Roche) was used for quantification of the PCR products. The PCR reaction for each cDNA was performed twice, using specific primers—one amplified only the upper band (exons 7, 8, and 9) and the other amplified only the lower band (exons 7 and 9). The PCR mixture (Roche) contained Taq DNA polymerase, reaction mix (buffer, SYBR Green I dye, dNTPs with dUTP instead of dTTP, 13 mM magnesium chloride) and 12.5 pmol of primers. The samples were run for 45 cycles of repeated 10 sec at 95° C., 10 sec at 61° C., and 10 sec at 72° C. Another reaction was performed, using specific primers for the GAPDH gene, that was used as an endogenous expressed control.

### Example 1

### Alternative Splicing of Exonized Alus

To study the alternative splicing regulation of exonized Alus, a dataset of exonized Alus was compiled from the human genome. Analysis of this dataset revealed that two positions along the inverted Alu sequence are most commonly used as 3′ splice-site (3′SS) in Alu exonizations: position 279 (‘proximal AG’) and position 275 (‘distal AG’). The relationships between two near AGs in a 3′SS were well characterized previously in the context of constitutive splicing. To pinpoint the sequence determinants by which the spliceosome decides which of the two possible AGs is selected in the context of alternative splicing, the exonized Alus that use either of these AGs were aligned to their ancestor.

FIG. 2 presents the 3′SS region of these instances wherein the numbers on top indicate the position relative to the distal 3′SS; “Gene name” is in accordance with RefSeq conventions; “Alu exon number” refers to the serial number of the Alu containing exon in the gene and “Subfam” refers to Alu subfamily type inferred using RepeatMasker (http://repeatmasker.genome.washington.edu/cgi-bin/RepeatMasker). Alignment is shown for the region near the two most prevalent 3′SS in the right arm of exonized Alu sequences (in the antisense orientation). Data for 29 exonized Alus, compiled from the results of our previous study (Sorek et al., ibid), as well as newly collected data from the literature, is shown. The 20 nucleotides presented are positions 290-271 in the Alu sequence, according to the numbering in ref. (Jurka et al., ibid). The two possible AG dinucleotides (distal and proximal to the poly-pyrimidine tract) are marked in bold. The selected AG dinucleotide, defining the end of the intron, is underlined for each exonized Alu. Selected AG dinucleotides were inferred, using alignments of expressed sequences to the human genome (Sorek et al., ibid). Marked by asterisk are additional Alu-exons found in the literature scan. Consensus sequences of subfamilies Sx and J appear in the first two rows, with positions differing between subfamilies marked in shaded dark gray.

Rows 30-32 represent the 3′SS of Alu sequences that their constitutive exonization was shown to cause genetic diseases: Alport syndrome (COL4A3), Sly syndrome (GUSB) and OAT deficiency (OAT). The mutation causing Alport syndrome is marked light-blue (position −7 G to T); exonizaiton in Sly syndrome and OAT deficiency resulted from mutations in the 5′SS. As shown in FIG. 2 the proximal AG is selected mostly in exonized Alus of S subfamilies (9 times out of 13), whereas the distal AG is mainly selected in exonized Alus belonging to J subfamilies (12 times out of 16). This differential usage of AG selection in Alu subfamilies is probably due to the polymorphism between the J and S subfamilies in position 277 (FIG. 2, shaded dark gray), which eliminates the distal AG in Alus of the S subfamilies. As a result, the proximal AG is selected. Although another polymorphism at position 275 creates a new distal AG in the S subfamilies, this new AG is 6 nucleotides downstream from the proximal AG, a distance that was shown to be out of the effective range for selecting a distal AG in constitutive splicing. Indeed, the cases where Alus of the subfamilies utilized the distal AG required mutations that shortened the distance between AGs back to 4 nucleotides (FIG. 2, shaded light gray). This indicates that when the range between the two AGs is 4 nucleotides or less, the distal AG is preferred, and when the distance is 6 nucleotides or more, the proximal is preferred.

However, in five cases (FIG. 2, rows 25-29), the proximal AG was selected, even though a distal AG existed less than 6 nucleotides in range; in all these cases, the G in position −7 (bold and italicized) was mutated either to A (2 cases) or T (3 cases). Remarkably, mutation in the same position in intron 5 of the COL4A3 gene leads to exonization of a silent intronic Alu. This Alu-exon is constitutively spliced, resulting in an Alport syndrome phenotype. This implies that the G in position −7 suppresses the selection of the proximal AG, causing a shift toward selection of the distal AG. When this G is mutated, the proximal AG is preferred. This is supported by the findings that GAG triplets at ends of introns are poorly cleaved in vitro and extremely rare in vivo.

### Example 2

### Proximal AG and Distal AG in Alternative Splicing of Exonized Alus

A mini-gene of ADAR2 gene (adenosine deaminase, involved in RNA editing) was cloned (FIG. 2, row 1). Previously, exon 8 of this gene (denoted also exon 5a in Slavov et al., Gene 299:83, 2002) was found to be an alternatively spliced Alu-derived exon, adding 40 amino-acids in frame to the protein. In this exon, the distal AG is used as the 3′SS.

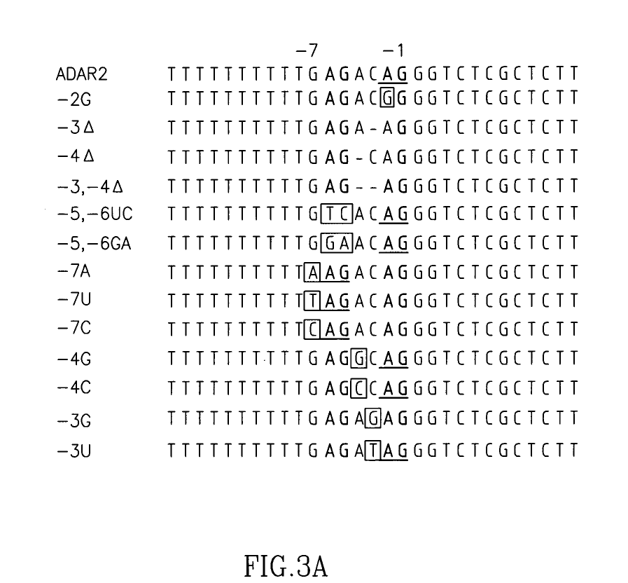

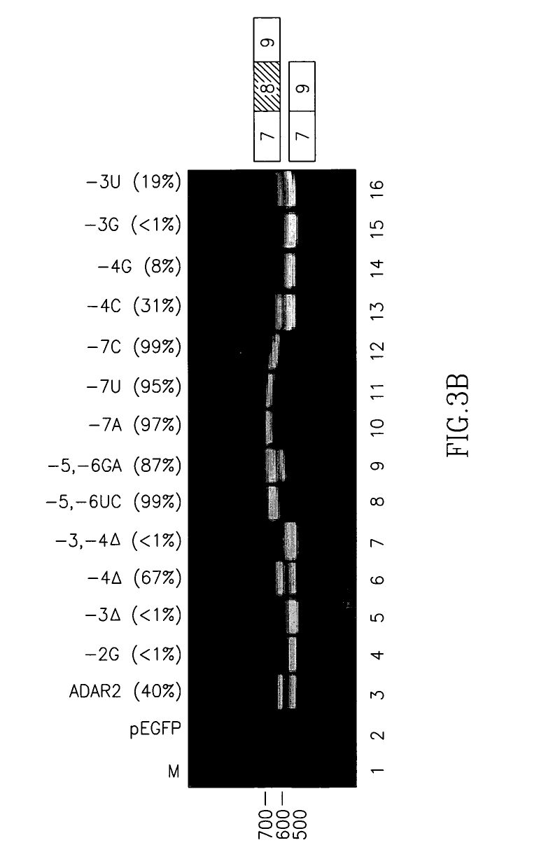

To characterize the relationship between proximal and distal AGs in the context of alternative splicing, a set (13) of mutations within the 3′SS was generated in the wild-type ADAR2 (FIG. 3A). The plasmid mutants were introduced into 293T cells by transfection, total cytoplasmic RNA was extracted, and splicing products were separated in 2% agarose gel following RT-PCR (FIG. 3B). The two possible mini-gene mRNA isoforms are shown on the right. Numbers in parenthesis indicate percentage of the Alu-containing mRNA isoform as determined by quantified RT-PCR (100% corresponds to the total of both mRNA isoforms). Identical results were also obtained with HeLa cells.

While the Alu-exon in the wild-type ADAR2 was included in 40% of the transcripts (FIG. 3B lane 3), replacement of the G in position −7 to A, U, or C (FIG. 3B lanes 10-12), had two effects. First, as predicted from FIG. 2, the replacement shifted the selection from the distal AG to the proximal one. Second, the replacement resulted in a shift from alternative splicing of the Alu-exon towards a nearly constitutive inclusion of the exon in the mature transcript. The results point to the important role of the G in position −7 in shifting the selection towards the distal AG, thus maintaining the alternative splicing of the Alu-containing exon. Mutation of that G will likely results in a constitutive inclusion of the Alu-exon and, thus, might cause a disease as occurred in the above-described case of Alport syndrome.

To check whether the proximal AG affects the selection of the distal AG, the proximal AG was mutated to UC or GA (FIG. 3B, lanes 8 and 9, respectively). The GA mutation resulted in a higher ratio of exon inclusion, reaching more than 85% inclusion, instead of 40% in the wild type. The UC mutation caused the splicing of the Alu-exon to become constitutive, possibly because it strengthened the poly-pyrimidine tract (PPT) that was originally 18 bases long (on average, the PPT length in exonized Alus was 19 bases+/−3). These findings indicate that the proximal AG presumably weakens the selection of the distal AG and is therefore required for maintaining alternative rather than constitutive splicing of the Alu-exon. To summarize, when the distal 3′SS is used, the G at position −7 suppresses the selection of the proximal AG, and the proximal AG maintains the alternative splicing.

We then sought to understand whether the nucleotide composition between the two adjacent AGs affects 3′SS selection and ratio of alternative splicing. The two AGs are separated by an AC dinucleotide (FIG. 2A). A deletion of both these nucleotides (position −3, −4) or only the C (FIG. 3B, lanes 5 and 7), resulted in an exon skipping, pointing to the importance of the C in position −3. Deletion or mutations of the A in position −4 to G or C changed the ratio between the two isoforms (FIG. 3B, lanes 6, 13 and 14). This indicates that position −4 also affects the inclusion ratio.

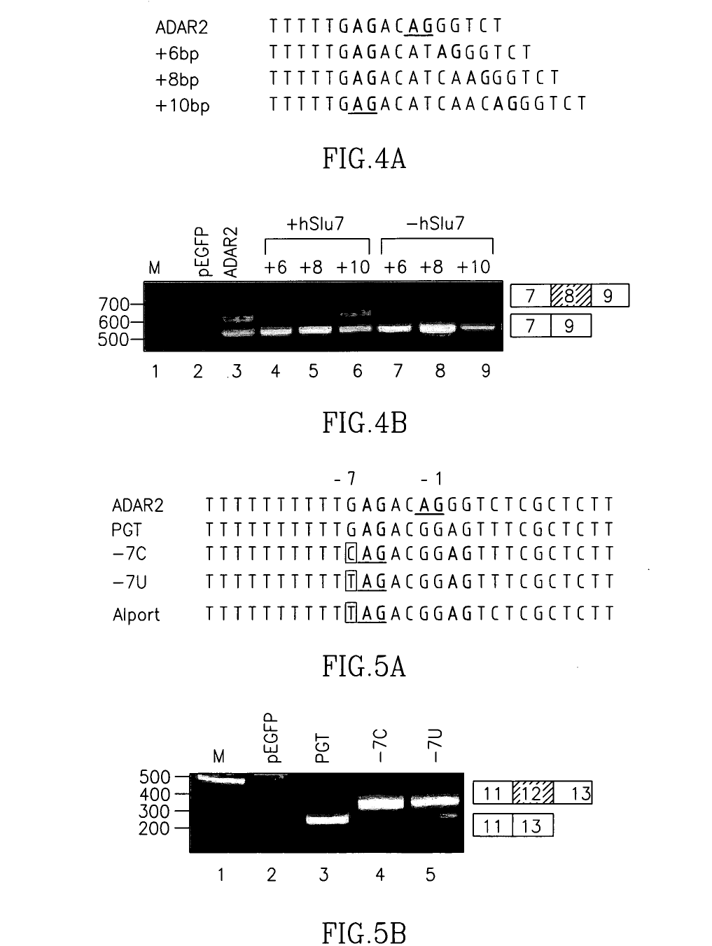

To test whether increased distance between the two AGs shifts the selection toward the proximal AG, additional nucleotides between the two AGs were introduced (FIG. 4A). Increasing the distance between the proximal and distal 3′SS to 6 or 8 nucleotides resulted in Alu exon skipping (FIG. 4B, lanes 7 and 8). However, when the distance between the two AGs grew to 10 nucleotides, a residual exon-inclusion was recovered in a little more than 3% of the spliced transcripts (FIG. 4B lane 9). In these transcripts, the proximal AG was selected, even though it was preceded by G (FIG. 4A).

### Example 3

### hSlu7 and Activation of Proximal AG in Alternative Splicing of Exonized Alus

We further examined whether hSlu7 (Human Synergistic Lethal with U5 snRNA), a second step splicing factor, is involved in the activation of the proximal AG (FIG. 4). This protein is known to be required for correct AG identification when more than one possible AG exists in the 3′SS region. Co-transfection of 293T cells with plasmids containing the insertion mutants and with hSlu7 (10 fold higher than endogenous hSlu7 concentrations) led to an increase in the selection of the proximal AG by 10-fold, reaching 32% inclusion when the distance between the proximal and distal AGs was 10 bases (FIG. 4B, lane 6). Presumably, hSlu7 activation of the weak splice-site may depend on the existence of a distal AG, because elimination of the distal AG (mutant −2G, FIG. 3) resulted in an exon skipping, which was not reversed by increasing the concentration of hSlu7. These results propose that the distal AG can affect the selection of the proximal one negatively, when the proximal is preceded by a G nucleotide. The proximal 3′SS can be selected when hSlu7 is present, and the efficiency of this selection is increased when the distal AG is found far enough from the splice-site (for example, 10 nucleotides into the exon). This, therefore, indicates that activation of the weak 3′SS (GAG) depends upon hSlu7 concentration and suggests a possible role of hSlu7 concentrations in alternative-splicing regulation.

Rows 17-22 in FIG. 2 show instances in which the proximal AG is selected, even though the distal AG is found 6 nucleotides downstream. However, the +6 bp mutant (FIG. 4B, lane 6) resulted in a total exon skipping. The above results suggest that these exonization instances might occur with high hSlu7 concentrations within certain cell-types, or high local concentration of hSlu7 within the subregion of the nucleus.

It was further assumed that, in normal conditions, these Alu exons would be skipped. For investigating this assumption, a gene encoding a putative glucosyltransferase (PGT) (FIG. 2, row 17) a mini-gene of its exons 11-13, including the introns in between was cloned (Alu exon being exon 12).

FIG. 5 presents splicing assays on wild-type and mutated putative glucosyltransferase (PGT). Panel (A) contains the following sequences (top to bottom): Wild-type Alu 3′SS of ADAR2; wild-type Alu 3′SS of PGT; Mutant Alu 3′SS of PGT; and Mutant sequence of COL4A3, causing Alport syndrome. Both potential AGs are marked in bold and the selected 3′SS is bold and underlined. The mutated position is shaded. Transfection was performed in HT1080 cell lines. Total RNA and RT-PCR was performed. The lanes in panel (B) presents the following: lane 1, DNA size marker; lane 2, vector only (pEGFP); lane 3, splicing product of wt PGT; and lanes 4-5, splicing products of mutated PGT minigenes, corresponding to the sequences in panel (A). The two possible mini-gene mRNA isoforms are shown on the right. The results were reproducible in 293T cell-lines.

Indeed, when PGT mini-gene was transfected into HT1080 and to 293T cell-lines, only a single mRNA isoform appeared, corresponding to Alu-exon skipping (FIG. 5B, lane 3). Repeating the same experiment using endogenous PGT mRNA also showed Alu-exon skipping.

Position −7 in the PGT mini-gene was mutated in order to test whether such mutation in a completely silent intronic Alu element would result in exonization. As seen in FIG. 5B (lanes 4 and 5), this point mutation was sufficient for activating the nearly constitutive inclusion of the Alu exon in the mature transcript. As indicated above, the same mutation in the COL4A3 gene activates a constitutive exonization of a silent intronic Alu, resulting in an Alport syndrome.

### Example 4

### Identification of 5′SS within Exonized Alus—in Silico Analysis

Data for 26 exonized Alus (Sorek et al. 2002, ibid) are shown in FIG. 6, wherein for each exonized Alu the 27 nucleotides spanning positions 146 to 172 according to the numbering in Jurka et al. (ibid) are shown, with Alu position 157 being the first position of the intron. The dinucleotides GT or GC that are selected as the 5′ss (defining the beginning of the intron) are in bold and italics. The 5′ss were inferred by alignment of expressed sequences to the human genome (Sorek et al. 2002, ibid) (table S1). The Alu consensus sequence appears in the first row; the position differing between Alu subfamilies S and J is framed in a box. Nucleotides that differ from the Alu consensus sequence are marked in bold; mutations that changed the dinucleotide CG to TG are marked in light grey; those for which this mutation creates a splice site are marked in dark grey; mutations that changed CG to CA are marked in italics. Row 26 represents the 5′ss of an antisense Alu sequence in intron 6 of CTDP1 gene in which a mutation from C to T at position 156 resulted in CCFDN syndrome. This mutation (marked black) led to the exonization of an intronic Alu sequence by activation of the 5′ss and the creation of an alternatively spliced Alu exon. Numbers on the top mark positions relative to the 5′ss. Gene names are as in RefSeq convention. The Alu exon number is the serial number of the Alu-containing exon in the related gene, and the Alu subfamily type was inferred with the use of RepeatMasker (http://repeatmasker.genome.washington.edu/cgi-bin/RepeatMasker).

The comparison of adjacent regions of the exonized Alus with the Alu antisense consensus sequence allowed the identification of nucleotide positions along the Alu sequence that need to be changed or remain conserved in order to enable Alu exonization. Two types of 5′ss were found: Introns that start with GC (FIG. 6, cases 1-4; SEQ ID NOS:33-36) and introns that start with GT (FIG. 6, cases 5-25; SEQ ID NOS:37-57). In the human genome, more than 98% of all introns begin with GT. The minority GC introns (˜0.7% of all introns) were asserted to be frequently involved in alternative splicing.

The most significant change observed between exonized Alus and their ancestral sequences was at position 156 (position 2 of the intron), where a mutation from C to T generates a canonical GT 5′ss at positions 157-156. This occurred in 21 of the 25 (84%) exonized Alus. In those cases in which positions 157-156 remained GC as in the ancestral sequence (rows 1-4), position 155 (representing position 3 of the intron) was found to be mutated from G to A.

In the ancestral sequence, CG dinucleotides are found in positions 156-155, 154-153, and 152-151 (FIG. 6, upper row). Since CG dinucleotides in Alu are frequently hypermethylated, and mutate 9.2 times more frequently than non-CG positions, one may attribute the formation of the GT 5′ss to the propensity of these sites to mutate from CG to TG. The prevalent mutations in positions 156, 154, and 152 are from C to T (light and dark grey, respectively), whereas in positions 155 and 151 the prevalent mutations are from G to A (italics).

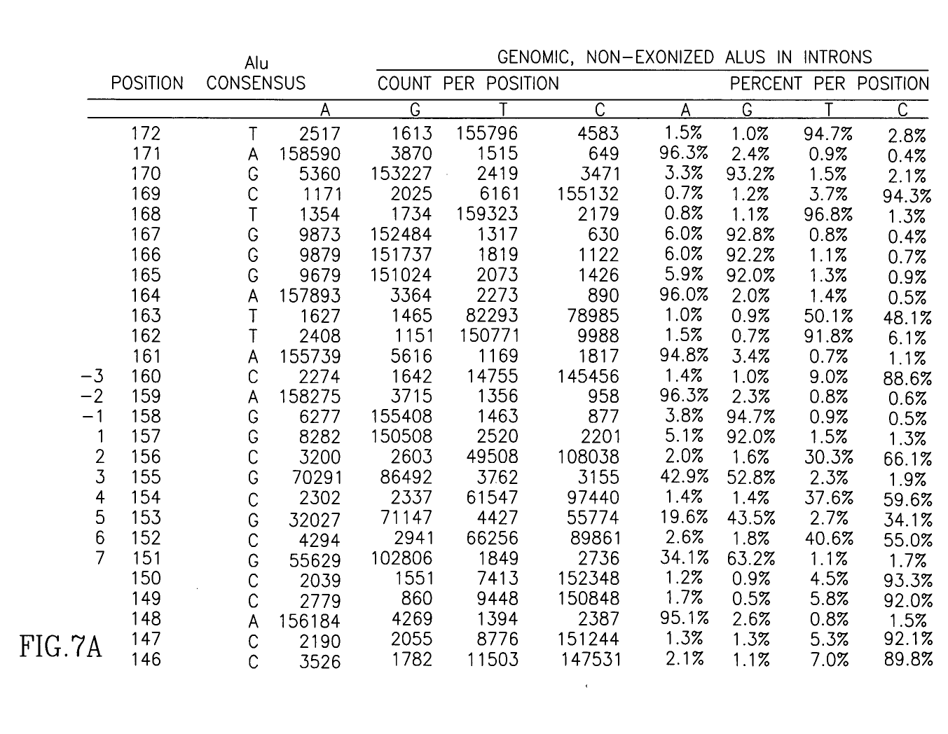

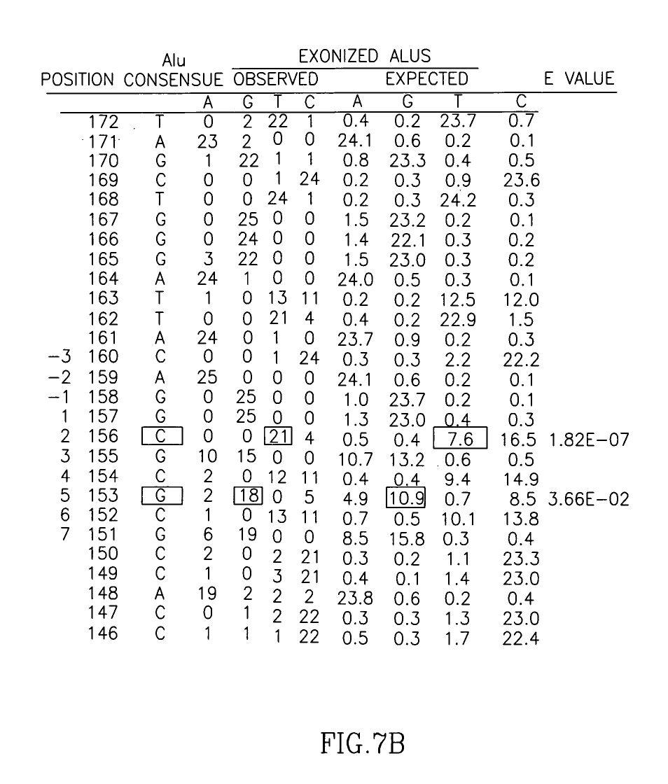

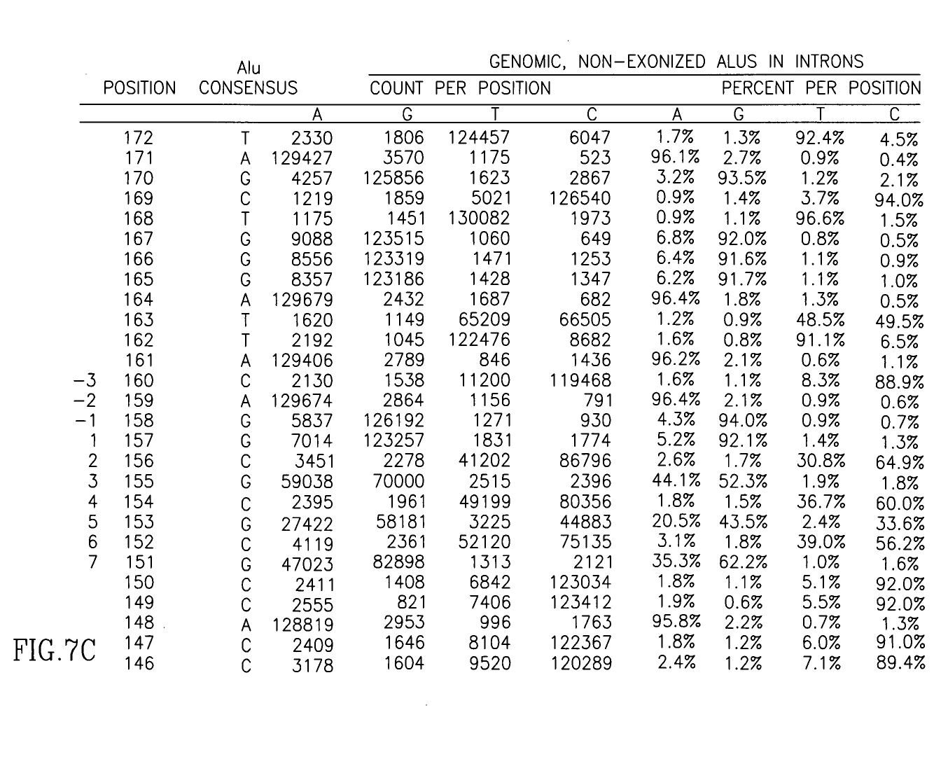

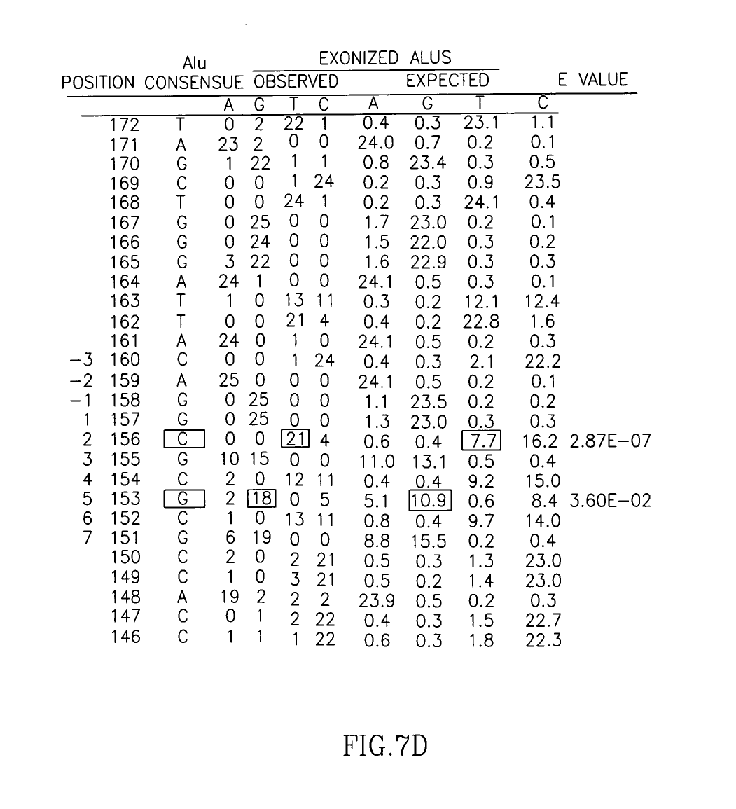

Some of the changes observed in FIG. 6 seem to be random (bold); others are presumably due to CG substitutions (light gray, dark gray, and italics). However, it is unclear which of these changes are important to the exonization of Alus and which represent inconsequential intronic substitutions. To pinpoint the positions that are important for exonization, we compiled a dataset of 166,276 full-length intronic Alus that are found in the antisense orientation in introns of human genes, and compared them to exonized Alus. For each position, the number of appearances of each nucleotide (count per position) is shown as well as the frequency of each base in the position (“percent per position”; FIG. 7A). By aligning each of these Alus to its ancestral sequence, we examined the percentage of each of the four nucleotides in each position along the Alu sequence (FIG. 7A; positions 172 to 146 of the antisense Alu consensus sequence are shown). We similarly examined the 26 Alu exons shown in FIG. 6 (FIG. 7B). Finally, we compared the nucleotide distribution for each position of the intronic Alus to those of the Alu exons, and looked for statistically significant deviations between the distributions (FIG. 7B). “Expected” profile was calculated by multiplying the profile matrix of non-exonized antisense intronic Alus from panel A by 25. Remarkable deviations between the observed and expected profiles are apparent in positions 156 and 153: in position 156, T is expected 7.6 times (marked gray), but observed 21 times (gray), indicating a strong tendency of this position to change from C to T in exonized Alus; in position 153, G is expected 10.9 times, but appears 18 times, indicating the importance of G in position 5 of the splice site.

Since sense and antisense-oriented Alus can be under different selective pressures in the human genome, we also compiled a dataset of 136,151 intronic Alus that are found in the sense orientation in introns and repeated the statistical comparison to exonized Alus. This yielded the same results, indicating positions 2 and 5 of the intron as significantly different between exonized and non-exonized Alus (FIG. 7C-D). This indicates that these are the most important positions in the creation of functional Alu-derived 5′ss. Therefore, Alus in the antisense orientation in introns are in fact “pre-exons” whose exonization requires only a small number of mutations.

Significantly, the same mutation, from C to T at position 156, of an antisense Alu sequence in intron 6 of the CTDP1 gene was found to be the cause of CCFDN (congenital cataracts, facial dysmorphism, and neuropathy) syndrome (FIG. 6, row 26; and SEQ ID NO:58). In this gene the G in position 153 is indeed conserved as predicted by our analysis. Previously, only mutations leading to the constitutive exonization of Alu elements were known to be deleterious, e.g., in Alport-syndrome type-I, Sly syndrome and ornithine aminotransferase (OAT) deficiency. CCFDN syndrome was the first reported case in which a mutation leading to the creation of an alternatively spliced Alu exon resulted in a genetic disease. Thus, the appearance of an aberrant Alu-containing spliced form may result in genetic disease even when the normal mRNA continues to be synthesized.

### Example 5

### Alternative Splicing Regulation in Exonized Alus

Following these results we wished to understand how the alternative splicing of these exonized Alus is regulated. In mRNA splicing, the 5′ss is recognized by three small nuclear RNPs (complexes of snRNA and proteins), one of which (U1) basepairs across the 5′ss junction (potential base-pairing between positions −3 to +6; for clarity, “−” and “+” indicate positions upstream or downstream of the 5′ss, respectively). This base-pairing is a prerequisite step for splicing in most introns. Although the importance of the U1 snRNA:5′ss base-pairing is well established in constitutive splicing, the function of this base-pairing in alternative splicing is only partially understood. We, therefore, set to understand the manner in which U1 affects the alternative splicing of Alu exons.

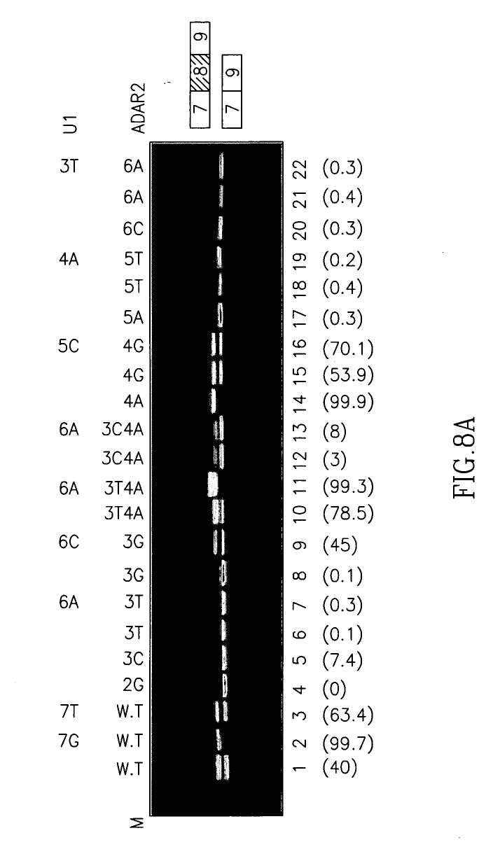

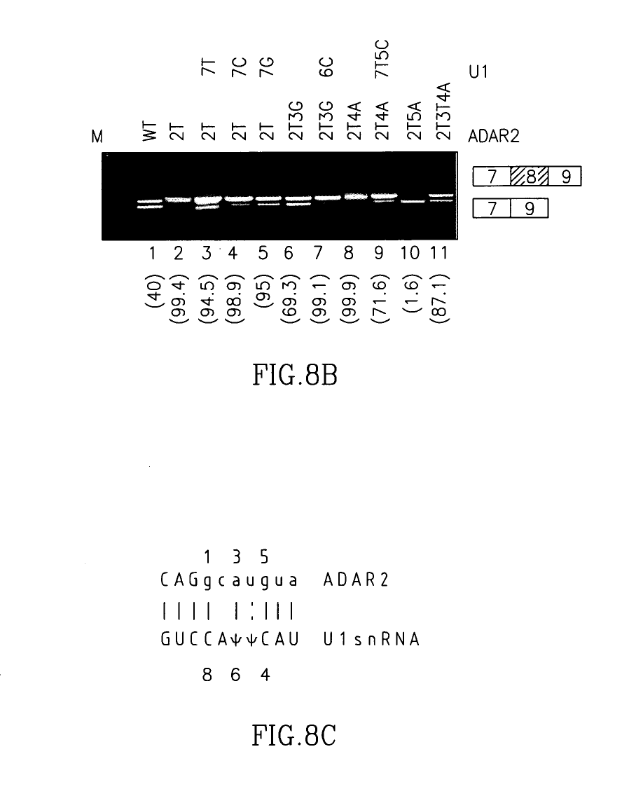

We used a mini-gene containing the genomic sequence of adenosine deaminase gene, ADAR2, from exon 7 to 9, in which exon 8 is an alternatively spliced Alu exon (Lev-Maor et al., ibid). The 5′ss of this Alu exon is of the GC type (FIG. 6, row 4; SEQ ID NO:36). To examine the function of the base-pairing between U1 and the 5′ss in alternative splicing, we transfected 293T cells with the ADAR2 mini-gene containing mutations in the 5′ss and complemented these mutations with co-trasfected U1 gene containing the appropriate compensatory mutation. Therefore, the cells contained exogenous and endogenous U1s that competed with each other to bind the 5′ss. Following transfection, cytoplasmic RNA was collected, and the splicing products were separated in 2% agarose gel after reverse transcription polymerase chain reaction (RT-PCR) enabling analysis of the splicing pattern of the ADAR2 mini-gene. We tested the effect of serial mutations on the splicing of the ADAR2 mini-gene when the first nucleotides of intron 8 are GC (FIG. 8A, representing GC 5′ss) or when they are mutated to GT (FIG. 8B, representing GT 5′ss). In FIG. 8A, the leftmost lane is the DNA size marker, lane 1 is the splicing products of WT ADAR2, and lanes 2-22 are splicing products of the indicated ADAR2 mini-gene mutated at the 5′ss. The two mRNA isoforms are shown on the right. Numbers in parentheses in the bottom indicate percentage of the Alu containing mRNA isoform as determined by TINA2 (100% corresponds to the total of both mRNA isoforms). Lane 11 contains two closely joined bands; the sequences of both were found to be identical and correspond to exon inclusion. This phenomenon may be attributed to migration of the over-expressed U1 together with the splicing product.

Our results indicate that the GC 5′ss maintains alternative rather than constitutive splicing directly because the C in position 2 of the 5′ss un-pairs with U1. This conclusion is supported by the fact that a compensatory mutation in U1 (A to G in position 7), which restores the base-pairing with position 2, results in the constitutive splicing of the exon (FIG. 8A, lanes 1-3; see FIG. 8C for wild-type (WT) U1:5′ss base-pairing).

As expected from our in silico analysis (FIG. 6, rows 1-4; SEQ ID NOS:33-36), when the 5′ss is of the GC type, an A in position 3 is essential for its proper selection; mutations to C, T or G led to Alu exon skipping (FIG. 8A, lanes 5, 6, and 8). This finding is in agreement with the weight metric of GC 5′ss where A is the prevalent nucleotide in position 3, and may suggest that an A at position 3 of the GC intron that forms A:T pairing (T=pseudo-uridine) with U1 is required to avoid two consecutive positions that un-pair with U1. A U1 containing a compensatory mutation restoring the base-pairing in position 3 of the 5′ss restores Alu exon inclusion (FIG. 8A, lane 9). In contrast, a T:A compensatory mutation in the same position failed to restore Alu exon inclusion (FIG. 8A, lane 7). This suggests that G:C rather than A:U pairing in position 3 of GC 5′ss contains the sufficient energy for U1 binding to the 5′ss. The failure of this A:T pairing to promote Alu exon inclusion is in contrast to the Ψ:A U1:5′ss pairing that is required for Alu exon inclusion. This might be related to the stabilizing effect of T on the backbone and the stem structure or to the dynamics it allows for a noncanonical interaction at the base of a stem.

Position 4 of the 5′ss affects the level of alternative splicing of the Alu exon. Mutation of that position from T to A, which strengthens the base-pairing with position 5 in U1, resulted in constitutive inclusion of the Alu exon (FIG. 8A, lane 14). Mutation of the same position from T to G increased the level of the Alu exon inclusion from 40% to 54%. The inclusion level increased to 70% following co-transfection with U1 containing a compensatory mutation that base-pairs with that G (FIG. 8A, lanes 15 and 16).

To understand whether or not a cooperative effect between positions 3 and 4 exists, we produced double mutants and tested their inclusion ratios. Mutations that introduced a mis-pairing between U1 and position 3 (A to T) but allowed better base-pairing of U1 with position 4 (T to A), restored alternative splicing of the Alu exon with a ratio of inclusion of 78% (FIG. 8A, lane 10). This splicing became constitutive when the cells where co-transfected with U1 containing a compensatory mutation that base-pairs with the T in position 3 (FIG. 8A, lane 11). Our results show that positions 3 and 4 are involved in controlling the level of exon inclusion in GC 5′ss. We note that position 3 in GC 5′ss may be T if position 4 is A; however, no such case was found in the set of exonized Alus, probably because such event would require two sequential transversions (from GC to TA), a combination of events that have a very low probability of occurrence in the genome.

The cooperative effect between positions 3 and 4 did not occur in double mutants in which position 3 was mutated to C and position 4 to A (FIG. 8A, lanes 12 and 13), implying that U, but not C, in position 3 of the 5′ss, can form a non-canonical Ψ:U pairing with Ψ in position 6 of U1. Indeed, Ψ:U base-pairing was recently reported to be important in position 4 of 5′ss in yeast splicing. The above results suggest a hierarchy in the strength of the pairing between U1 and position 3 of the 5′ss: Ψ:A>Ψ:U>Ψ:C. This hierarchy seems to determine the level of alternative splicing.

We further studied the importance of positions 5 and 6 in GC 5′ss. Mutations in these positions resulted in the total skipping of the Alu exon, which compensatory mutations in U1 failed to restore (FIG. 8A lanes 17-22). This suggests that other splicing factors also recognize these positions.

To study the regulation of Alu exons with GT in their 5′ss, we mutated the C in position 156 of the ADAR2 Alu exon to T, creating a GT 5′ss (FIG. 8B). This mutation resulted in a shift from alternative to constitutive inclusion of the Alu exon (FIG. 8B, lanes 1-2), further supporting our conclusion that the weak base-pairing with U1 maintains the alternative splicing of the GC 5′ss. Co-transfection with U1 that un-pairs with the T at position 2 had only a minor effect of ˜5% exon skipping (FIG. 8B, compare lane 2 to lanes 3-5), presumably reflecting the effect of U5(p220) binding to that nucleotide. In addition, as predicted from our bioinformatics (in silico) analysis (FIG. 7), G in position 5 of the intron is essential for the alternative splicing of Alu exons, as a G to A mutation in that position resulted in total Alu exon skipping (FIG. 8B, lane 10).

In contrast to the GC 5′ss, which required an obligatory A in position 3, mutation of that position to G in the GT 5′ss reduced the splicing from constitutive to alternative, but did not eliminate exon inclusion entirely (FIG. 8B, compare lane 2 to 6). This probably stems from the fact that GT introns are generally stronger 5′ss than GC introns, and a G in position 3 of the GT 5′ss can form a G:Ψ pairing with U1. Co-transfection with U1 containing a compensatory mutation that base-pairs with the G in position 3 restored a constitutive splicing, suggesting that the base-pairing of that position with U1 is a main factor affecting the alternative splicing ratio (FIG. 8B, lanes 6-7 and also FIG. 8A, lane 10). A similar preference for A over G in position 3 was also observed in other 5′ss where the base-pairing between U1 and the 5′ss was sub-optimal.

To test the importance of position 4 in the GT 5′ss, we mutated it to A, which base-pairs with the WT U1. This maintained the constitutive inclusion of the Alu exon (FIG. 8B, lane 8). When a mutated U1 that un-pairs with position 4 was co-transfected, the splicing became alternative (FIG. 8B, lane 9). A return to alternative splicing was also observed when an additional mutation, which un-pairs with U1, was introduced in position 3 (FIG. 8B, lane 11). This further supports the results from the GC 5′ss analysis, which showed that the nucleotide composition in positions 3 and 4 affects the delicate balance of skipping or inclusion of alternatively spliced exons.

From these serial mutations in the 5′ss and the compensatory mutations in U1 we conclude the following: (i) U1:5′ss base-pairing is involved in both GC and GT 5′ss selection. (ii) The alternative splicing of ADAR2 Alu exon is maintained due to the un-pairing of U1 with position 2 of the 5′ss. (iii) The nucleotide composition of positions 3 and 4 in the intron, both in GT 5′ss and in GC 5′ss, control the delicate skipping/inclusion ratio depending on the canonical/non-canonical base-pairing of these positions with U1. (iv) An A in position 3 of the intron is more important in GC 5′ss than in GT 5′ss, possibly due to the need to avoid two successive non-pairing positions. (v) G in position 5 is essential for the selection of 5′ss in the two types of introns.

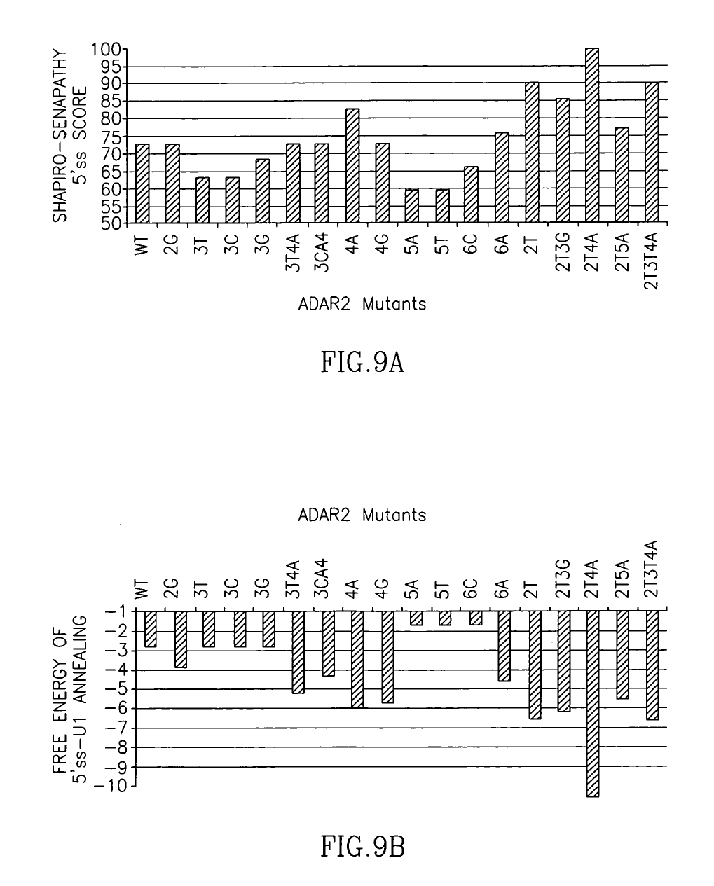

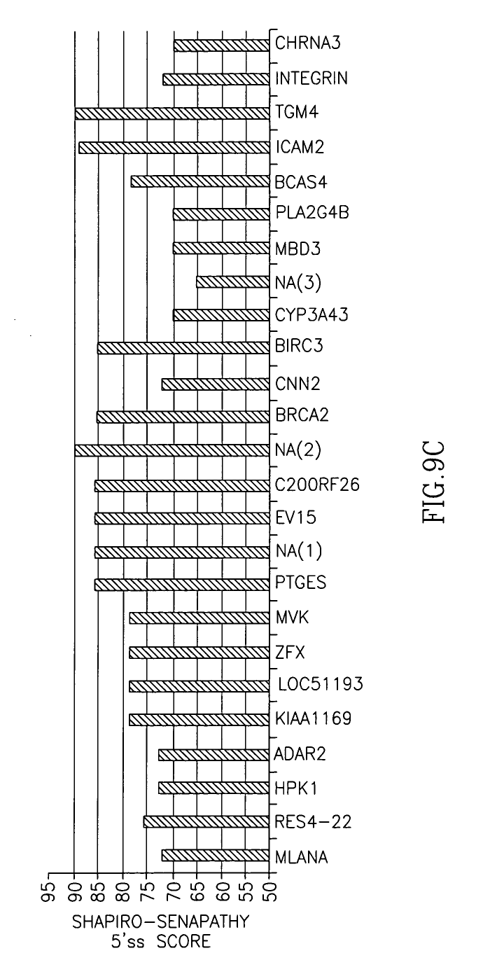

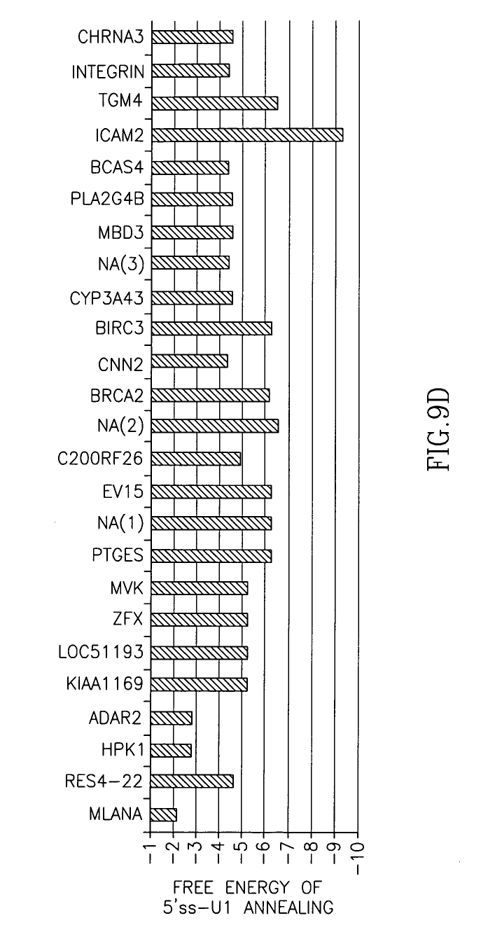

The results for position 4 of the 5′ss indicate that when this position is mutated to A the ADAR2 exon becomes constitutive. However, there are two cases among the 25 alternatively spliced Alu exons in our compilation that contain A in position 4 (FIG. 6, rows 2 and 22; SEQ ID NOS: 34-57). A similar inconsistency is observed for the mutation of position 6 from T to C, which makes the ADAR2 exon inactive, but is found in several exonized Alus (FIG. 6). To further examine this inconsistency we calculated splice site scores and free energy of U1:5′ss binding (ΔG) in exonized and mutated Alus (FIG. 9). Free energy was calculated using the software mfold version 3.1 and according to Carmel et al., (RNA, 10:828, 2004). The splice site score is a measure of how “close” the splice site is to the consensus sequence profile of 5′ss (Shapiro and Senaphthy, ibid). The free energy is a measure of the U1:5′ss binding strength, taking also into consideration the differences between G:C, A:U, and G:U base pairing and stacking energy—lower ΔG values stand for stronger binding and might indicate higher exon inclusion/exclusion ratio (Carmel et al., ibid).

When position 6 in ADAR2 is mutated to C, the 5′ss score is reduced from 73 to 65 (FIG. 9A, mutant 6C), and the free energy is increased from −2.8 to −1.7 which indicates that binding is inefficient (FIG. 9B, mutant 6C). In exonized Alus where position 6 is C the 5′ss score can also get as low as 65 (for example, NA(3) in FIG. 9C), but the U1:5′ss free binding energy in these exons is never higher than −4.0 (FIG. 9D, see for example ZFX, MVK, NA(3) and MBD3). The lower ΔG stands for more efficient U1:5′ss binding, which explains why these exons are still recognized.

When position 4 in ADAR2 is mutated to A, the 5′ss score increases from 73 to 83, and the AG becomes −6.0 (FIG. 9A-B, mutant 4A), which is in agreement with the fact that this mutation results in a constitutively spliced exon. In the exonized Alu in gene RES4-22 position 4 is A, but the 5′ss score is only 75 (similar to the score of the WT alternative ADAR2) and the AG is −4.6 (FIG. 9C-D). This can explain the fact that the Alu exon of RES4-22 is alternatively spliced. In the Alu exon of ICAM2 position 4 is also A, and the 5′ss score and ΔG indicate a very high affinity to U1. Still, that Alu exon is alternatively spliced, which might indicate that sequence elements other than the ones in the actual 5′ss are involved in its regulation.

Overall, the 5′ss score for exonized Alus was similar to that for non-Alu alternative cassette exons, averaging 78.3 in the 25 Alu exons and 79.8 in the set of 243 non-Alu cassette exons from Sorek and Ast (Genome Res. 13:1631-7, 2003). The free energy value was also similar between the sets, with an average of −5.08 in the 25 Alu exons and −5.29 in the non-Alu cassette exons.

The results presented here and in Lev-Maor et al. (ibid) indicate that the sequence composition of both 3′ss and 5′ss determines the alternative splicing ratio of Alu exons. In addition, our statistical comparison between intronic Alus and exonized ones, shows that only two positions along the Alu sequence substantially differ between intronic and exonized Alus; both in the 5′ss sequence (FIG. 7). These results imply that only the sequences of the 3′ and 5′ss of the Alu exons are important for exonization. This conclusion is supported by the finding that, so far, all the mutations leading to exonization of intronic Alus that cause genetic disorders were only found to affect 5′ss or 3′ss sequences (references in FIG. 6 and in Lev-Maor et al., ibid).

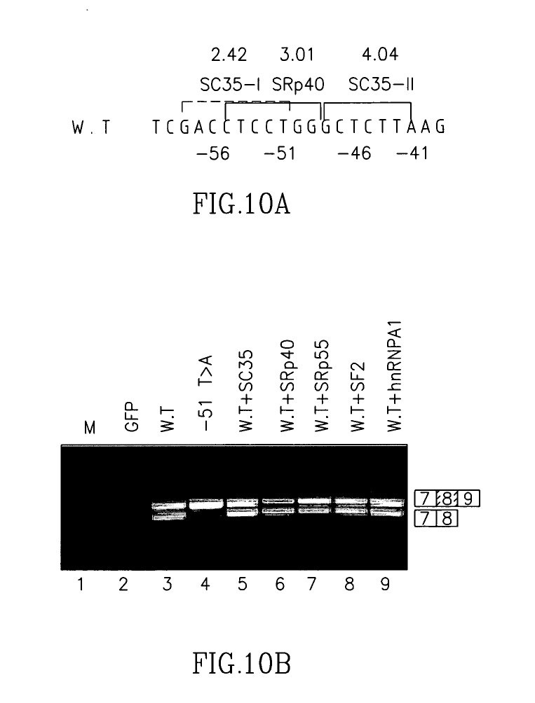

Still, it is possible that the splicing of Alu exons is regulated by splicing enhancers or silencers residing outside the splice sites. We used the ESE finder to search the sequence of the Alu exon of ADAR2 for such potential sequences. Seven potential binding sites for SR proteins were found, 3 of them in close proximity (FIG. 10A). SR proteins are known to be involved in alternative splicing regulation through binding to short sequences on the RNA molecule. The score of all potential SR binding sites was low, but such sites can still be functional. To test whether these sites are functional we serially mutated positions −48 to −54. We found no effect on the splicing pattern of the Alu exon, indicating that these potential ESEs are not functional. Mutations in position −11, embedded within another weak potential ESE, gave similar results.

To examine whether other sites are involved in the regulation of the Alu exon splicing, we co-transfected 293T cells with the ADAR2 mini-gene and various plasmids containing the most abundant splicing regulatory proteins (SR proteins and hnRNP A1, the later was shown to promote exon-skipping (FIG. 10B lanes 5-9). In principle, if one of these proteins was involved in the regulation of Alu exon splicing, then increasing its nuclear concentrations might affect the skipping/inclusion ratio of the Alu exon. This was not the case: None of these proteins affected the alternative splicing of the Alu exon. This suggests that the Alu exon of ADAR2 has no splicing enhancers or silencers that can bind to one of these proteins and regulate the Alu exon splicing, but we cannot rule out the possibility that other splicing regulatory proteins which are not part of the panel in FIG. 10, affect the inclusion/skipping ratio of this exon. As a control to that experiment we artificially created a strong binding site for SC35, by mutating T in position −51 to A and thus increasing the ESE score from 2.4 to 4.2. This mutation led to a shift towards exon inclusion, indicating that, in principle, SR proteins have the potential to modulate the splicing of Alu exons, but are presumably not involved in the case of the exon under study (FIG. 10B lanes 3-4). This further supports the analysis in FIG. 7, implying that in the general exonization process of Alu exons there is no selective pressure for the creation or lost of a splicing regulatory sequence/s located outside of the splice sites and suggesting that only the sequences of the splice sites are involved in that process.

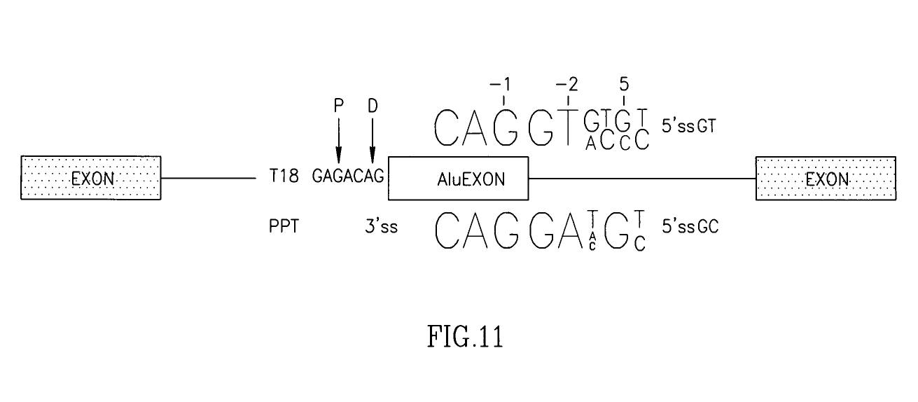

The sequence elements needed for the creation of an alternatively spliced Alu exon are depicted in FIG. 11. The prevalent 3′ splice site (3′ss) is found in position 275 of the antisense Alu, and is composed of a polypyrimidine tract (PPT) averaging 18 bases in length, followed by a 3′ss motif of GAGACAG containing a proximal and a distal AG (P and D, respectively) (Lev-Maor et al., ibid). The prevalent 5′ss can either begin with GT or with GC. Pictograms (http://genes.mit.edu/pictogram.html) depict the profiles of 21 GT-exonized and 4 GC-exonized Alus, above and bellow the box, respectively.

### Example 6

### Alu Exonization in ACAD-9 Gene is Specific to Ovarian Cancer

The results presented in the present application demonstrate that an activation of an intronic Alu sequence (silent Alu sequence never spliced in) can be induced either by point mutations in certain positions along the Alu sequence, or by changing the concentration of the splicing regulatory proteins in the cell. Both high rate of mutations and aberrant concentration of splicing regulatory proteins are implicated in cancer cells.

If alternative RNA splicing isoforms are associated with specific cancer cell types, then they could potentially serve as diagnostic markers for cancer. This idea was tested herein. Alternative splicing isoforms of ACAD-9 gene (e.g. SEQ ID NO:64) containing Alu exons (e.g. SEQ ID NO:63) were classified as tumor or normal, based both on the source of the mRNA used to construct the relevant cDNA library and whether the isoform was present at a higher frequency in tumor cDNA libraries than in normal cDNA libraries. This analysis showed that 24 out of the 61 alternative RNA-splicing isoforms containing Alu exon were significantly associated with tumor libraries. The alternative-splicing isoforms were associated with ovary, brain, head-neck, lung, kidney, and prostate cancers. A partial list presented in Table 1.

TABLE 1
Alu exon associated with tumor libraries

| | EST that skipped Alu | | EST that included Alu | | | | Tissues that |
|---|---|---|---|---|---|---|---|
| Gene | exons | | exons | | Alu | | included the Alu |
| no. | ID | No. of EST | ID | No. of EST | Family | Annotation | exon |
| 1 | HSAJ4875 | 36 | AA225691 | 10 | Sp | Putative | Normal fetal lung |
| | | | | | | glucosyl- | fibroblast; prostate; |
| | | | | | | transferase | fetus-testis + lung + B |
| | | | | | | | cell; brain |
| | | | | | | | glioblastoma; |
| | | | | | | | multiform |
| 2 | AA210960 | 6 | AK021447 | 3 | Sg | unknown | Embryo 10 w old; |
| | | | | | | | mainly head; fetal |
| | | | | | | | brain |
| 3 | AF003924 | 6 | AW954573 | 3 | Sg | Zinc finger | Normal infant brain |
| | | | | | | protein | |
| | | | | | | ANC_2H01 | |
| 4 | AK022568 | 16 | BE898836 | 7 | Jb | ACAF-9 gene | Ovary; adeno- |
| | | | | | | | carcinoma cell line |
| 5 | ABO18010 | 53 | AW381165 | 4 | Jb | Membrane | Head-neck carcinoma |
| | | | | | | glycoprotein | |
| | | | | | | 4f2 heavy chain | |

To validate the computation-predicted alternative RNA-splicing isoforms, we selected two genes: ACAD-9 (AF327351) and TIF-IA.

ACAD-9 belongs to the family of Acyl-CoA dehydrogenses which are mitochondrial enzymes catalyzing the initial rate-limiting step in the b-oxidation of fatty acyl-CoA. The reaction provides main source of energy for human heart and skeletal muscle. The ACAD-9 gene contains 18 exons mapped to chromosome 3q26, it shares 65% similarity with VLCAD (very long chain acyl-CoA dehydrogense). Deficiency of this protein may leads to severe human genetic diseases, for example: sudden death in infancy, infantile cardiomyopathy, hypoketotic hypoglycemia, hepatic dysfunction. The protein is ubiquitously expressed in most normal tissues with high expression in heart, skeletal muscles, brain, kidney and liver and also in most cancer cell lines.

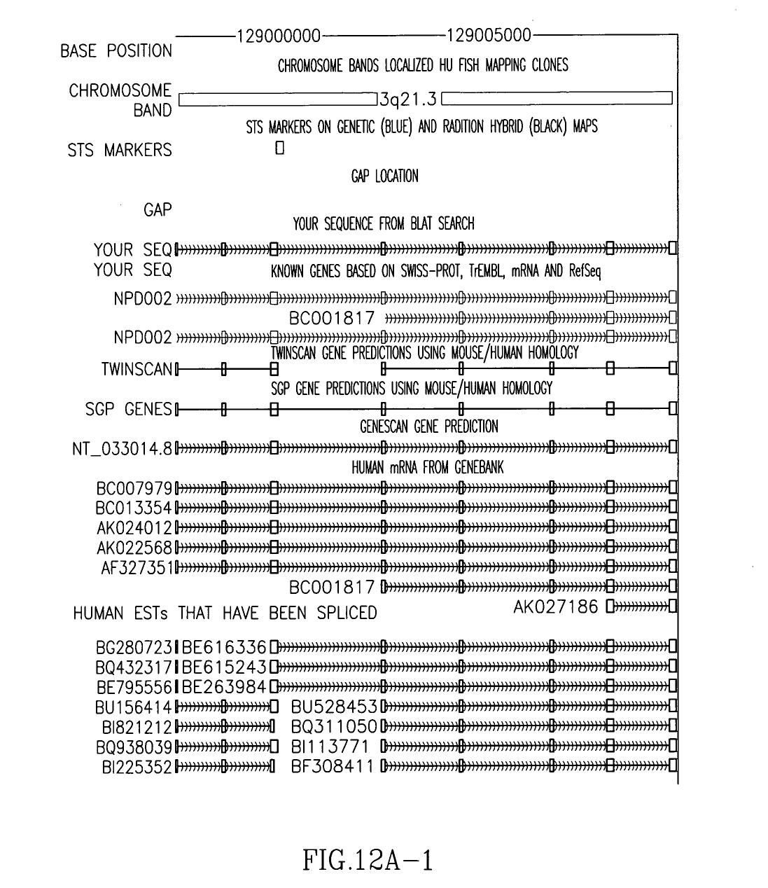

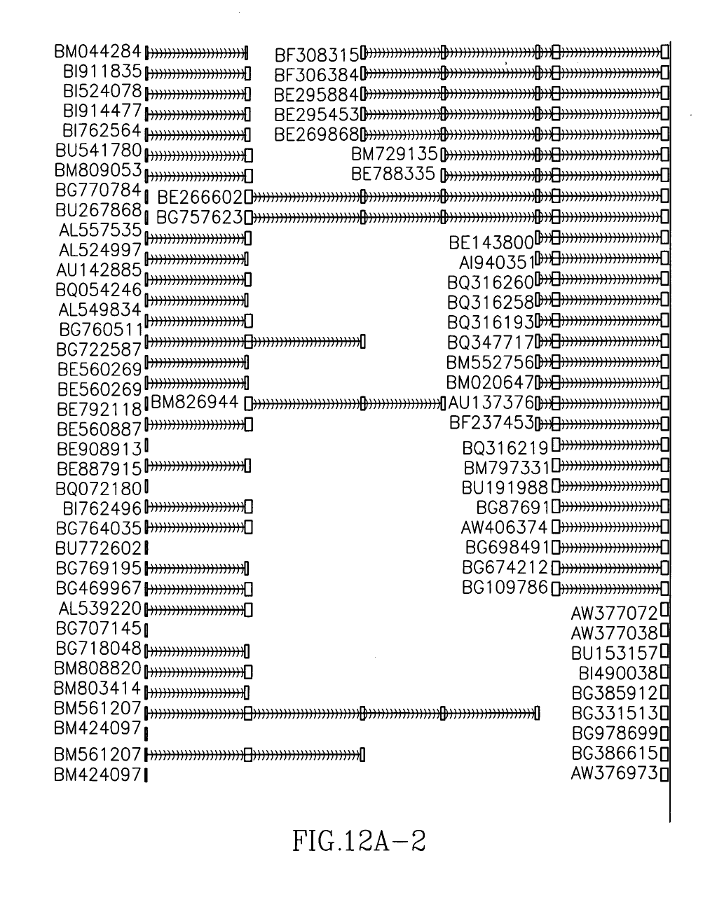

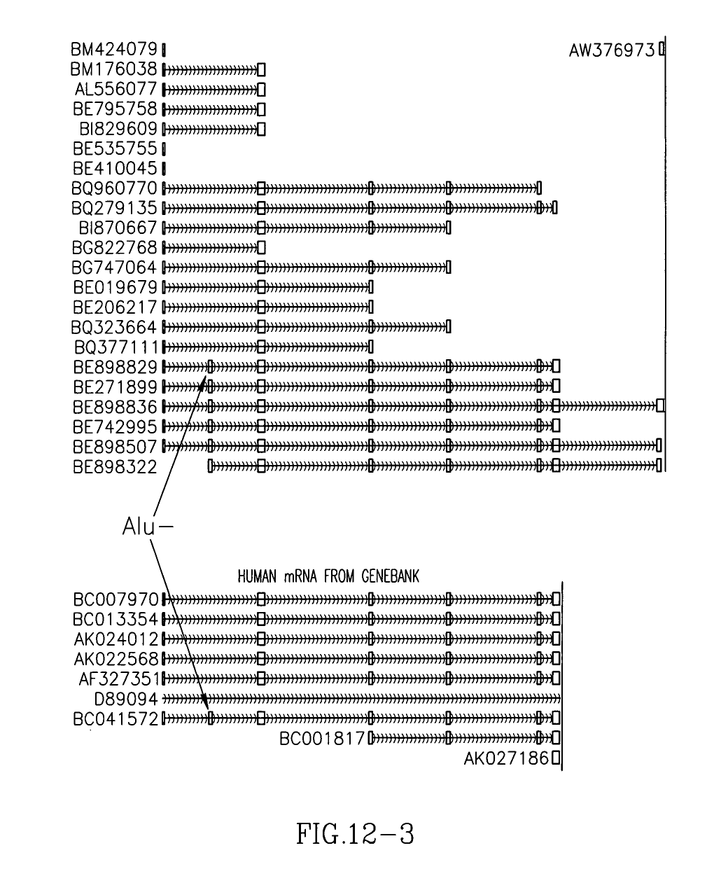

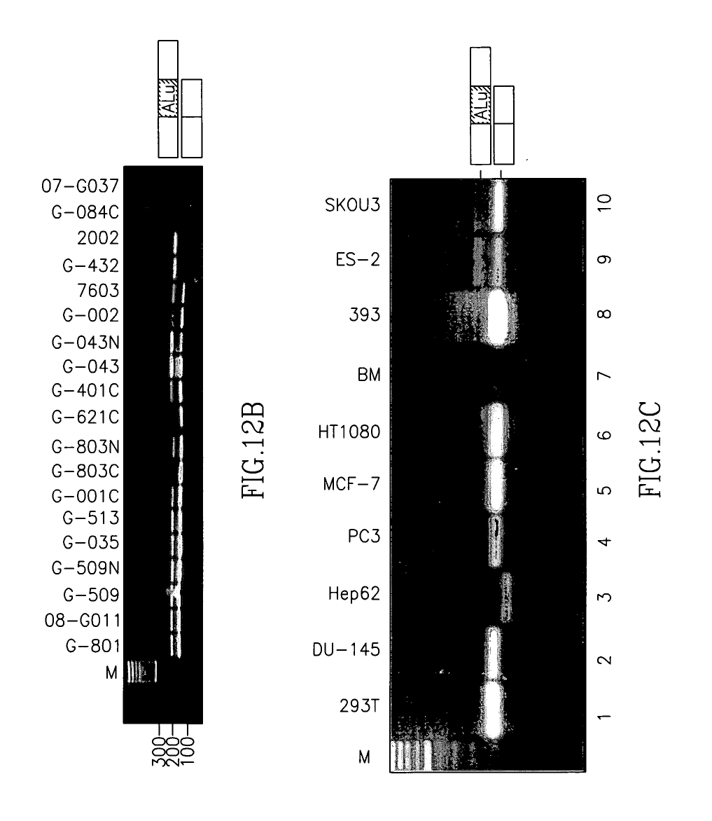

For ACAD-9 gene we identified 7 ESTs (Expressed Sequence Tag), from 2 different libraries, containing the Alu exon, all 7 ESTs were detected exclusively in human ovarian cancer. The exonized Alu insert is a 76 bases exon originated from intronic Alu in intron 4 (FIG. 12A; SEQ ID NO:63). The Alu exon causes a frame-shift that removes three out of five putative acyl-CoA dehydrogense domains.

The alternative splicing pattern of the ACAD-9 gene in ovarian cancer was examined in 16 tissue samples of human ovarian cancer (obtained from Gynecologic Oncology Group) and 3 samples of “normal” tissues (derived from the humans bearing ovarian cancer). Total cytoplasmic mRNA was isolated and cDNA synthesis was performed using primers that were designed to detect regular- and alternative-splicing products (directed to the flanking exons of the Alu element): Reverse primer—5′ CACTGATCCATCAGAATCAACG 3′ (SEQ ID NO: 61) and Forward primer—5′ GCACTACATCCTCAATGGCTC 3′ (SEQ ID NO: 62). Reaction products were analyzed by agarose gel electrophoresis (FIG. 12B). Our results indicate that 15/19 samples contain an mRNA isoform with the Alu exon, of which two contain only the Alu-inclusion form (G-432 and 2002). However, this pattern was not observed in non-ovarian cancerous cell lines including lymphoid and leukemia cancers cells, although that in ovarian cancer cell line we detected low level of Alu-inclusion with respect to other cancer tissues (FIG. 12C, and also a comparison between panels C and B, respectively). The results suggest that this Alu exonization is specific to ovarian cancer.

The fate of ACAD-9 gene, in the level of genomic DNA, mRNA and proteins in is examined in a collection of 100 ovarian cancer tissues, normal tissues, and uterus cancer tissues.

### Example 7

### Exonization of an Intronic Alu Sequence in TIF-IA is Specific to Leukemia and Lymphoma

TIF-IA is a 75 kDa protein that plays a major role in directing growth-dependent regulation of ribosomal DNA transcription. The amount or activity of TIF-IA fluctuates in response to cell proliferation. TIF-IA is also an essential initiation factor of RNA polymerase I (Pol I) transcription in growing cells. The fact that regulation of ribosomal gene transcription is a crucial element in controlling cell growth, and normally balances the effects of growth factors to prevent uncontrolled cell proliferation, appoints TIF-IA as a fundamental functional protein in adapting cellular biosynthetic activities to cell growth.

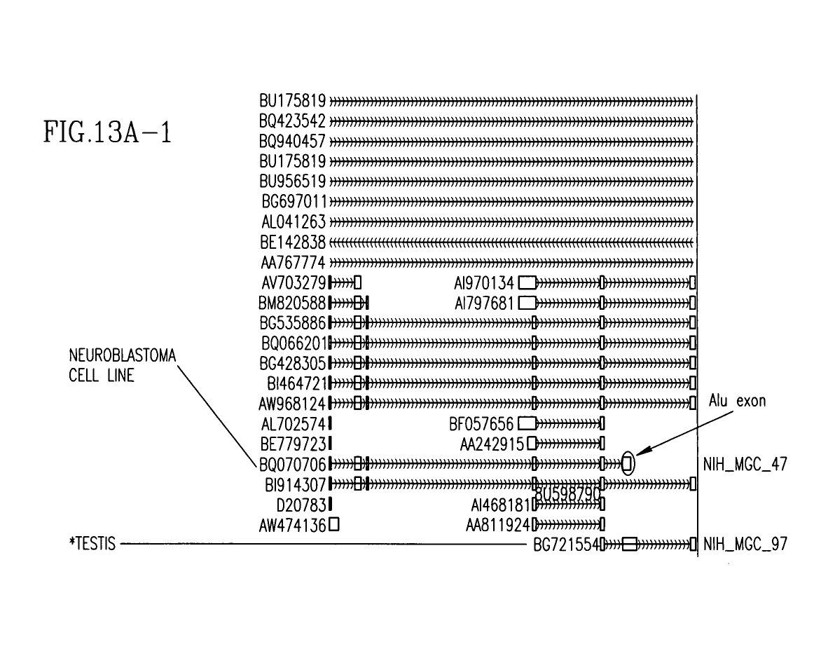

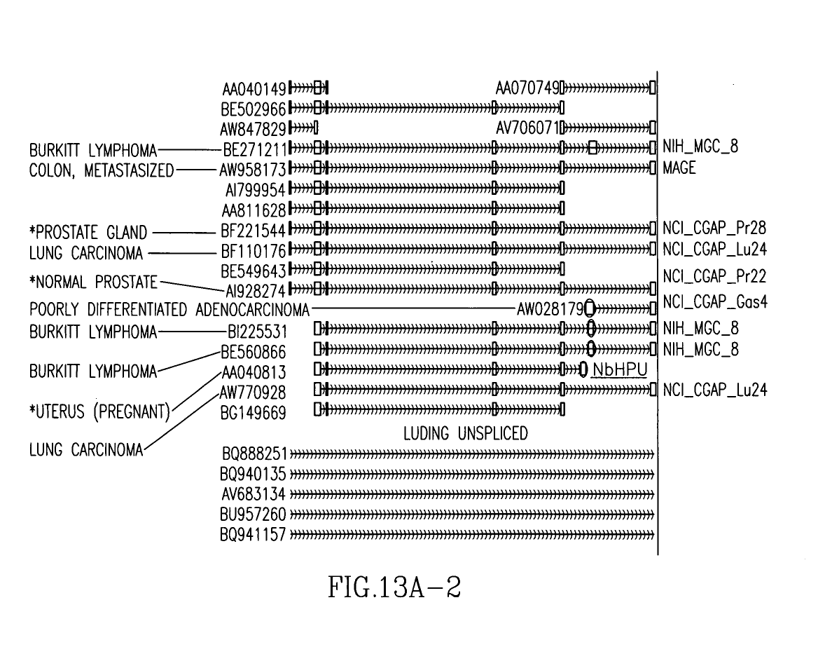

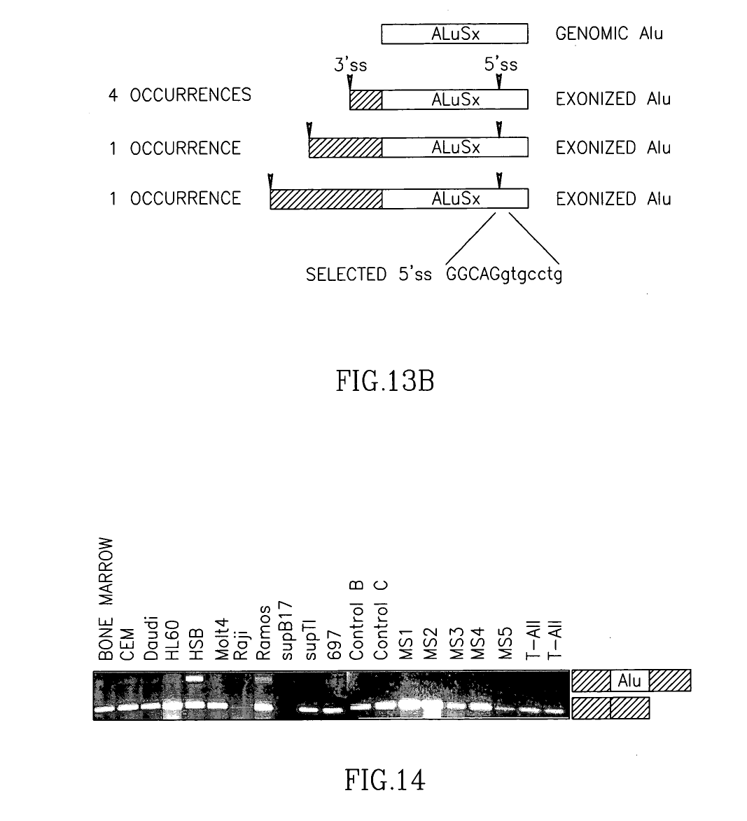

The TIF-IA gene was found to have six mRNA isoforms which contain an Alu exon. These TIF-IA isoforms were detected mostly in various cancers as shown in FIG. 13. A BLAT alignment of the human TIF-IA gene with the ESTs and mRNA using UCSC genome browser (FIG. 13A) identifies seven ESTs comprising Alu-derived exon (circled and mark by an arrow). These ESTs are originated from different cDNA libraries (on the left), cancer library and normal library (star). The exonized Alu is originated from different tissues and cell-lines, whereas the predominant cancer involved in this exonization is Burkitt Lymphoma (3 ESTs) and Lung Carcinoma (2 ESTs) in addition to neuroblastoma. Interestingly a possible common denominator of these cancers is the involvement of the myc family proto-oncogene.

Exonization of intronic Alu element derived from the Sx subfamily is presented schematically in FIG. 13B. The first line represents the intronic Alu element of the Sx subfamily. Three different types of exonization were identified in silico: (i) the same 5′ splice site (5′ss; or donor site) was selected for all three types of exons whereas three different 3′ splice sites (3′ss; or acceptor site) were selected. The position of the 5′ss and 3′ss are marked by arrowheads. The Alu sequence contributed the 3′ half and the 5′ss for all of these exons. While the 5′ half and the 3′ss are contributed by non-Alu sequences (marked by diagonal lines). The number of times that each type of exon was found in the dataset is shown on the left. The 5′ss sequence is shown at the bottom of the figure, uppercase and lowercase letters are exon and intron sequences, respectively.

The function of the Alu exon(s) in TIF-IA is unknown. The most prevalent Alu exon (4 occurrences) is 180 bp long and does not posses a stop codon, therefore after splicing, the sequence translates to a protein with an additional domain whose function is yet unknown. Bioinformatics analysis reveals potential phosphorylation sites on this region.

The alternative splicing pattern of the TIF-IA gene in different leukemia (mostly lymphatic) cell-lines was examined. Total cytoplasmic mRNA was isolated and cDNA synthesis was performed using primers that were designed to detect regular- and alternative-splicing products (directed to the flanking exons of the Alu element; Forward primer: GTC GCG TTA GTT CGG CCC (SEQ ID NO:59); Reverse primer: CTT CAG CAA GAC TTC TGT CAC (SEQ ID NO:60). Reaction products were analyzed by agarose gel electrophoresis (FIG. 14). In all samples a faint product containing the Alu exon (3% of the total mRNA from this gene, as examined by TINA2) was detected, and the major mRNA product skipped the Alu element. However, in two cell lines HSB (a T-cell ALL cell line) and Ramos (a Burkitt lymphoma cell line) a substantial portion of the mRNA products of the TIF-IA show inclusion of the Alu exon −48% and 12%, respectively. The identity of the PCR products was confirmed by DNA sequencing.

### Example 8

### Screening Patients For Alu-Containing TIF-IA mRNA

The presence of this RNA and is sought by screening the number and type of the mRNA isoforms generated from this gene on at least 100 primary samples obtained, with consent, from patients suffering from childhood acute lymphoblastic leukemia. These samples are collected as a part of the Israeli National Study of childhood leukemia treated on the international BFM protocol. Specificity is obtained by of collecting and performing a similar analysis on 100 samples of tissues from ovarian and uterus cancers on samples of normal peripheral blood cells or bone marrow cells (collected from donors for stem cell transplantation). The examination is carried out as follows:
(a) mRNA from the cells/tissues is purified, and RT-PCR is applied using primers designed to detect regular- and alternative-splicing products of each mRNA isoforms.
(b) The RT-PCR product is isolated and then sequenced, using Lev-Maor et al., (ibid) protocols. The sequence of each isoform is aligned (BLAST) against the human genome sequence to determine the mRNA splicing pattern according to Sorek and Ast (ibid) protocols.
(c) The correspondent genomic region of this gene from HSB and Ramos cell lines (FIG. 14), and from patients that show aberrant pattern of mRNA splicing is amplified by PCR and sequenced to enable determining whether the appearance of additional isoforms is due to mutations that effect mRNA splicing. In cases where no mutation is found, the concentration and distribution of several splicing-regulatory proteins (such as SR proteins) in the cells is examined, using immunoblotting and immunostaining techniques. As a control (standard), the pattern of mRNA isoforms of these genes in ovarian and uterus cancer is examined indicating whether the activation of these isoforms in these cancers is specific. The correlation between the aberrant isoform expression and patient characteristics is documented for various parameters including, without limiting to, demographics, WBC at presentation, cytogenetics, immunophenotype and response to therapy.

### Example 9

### The Effect TIF-IA mRNA Containing an Alu Exon on Different Cellular Activities

To examine the effect TIF-IA mRNA containing an Alu exon on different cellular activities TIF-IA cDNA is cloned with and without the Alu exon. The ultimate goal of the TIF-IA cDNA cloning is to use it as the basis for functional studies in cell lines. Total RNA is isolated from several lymphoblastic cell lines known to express the two mRNA isoforms. cDNA is produced by reverse transcribing the mRNA and large amounts of cDNA encoding both isoforms of TIF-IA are obtained using PCR. The amplified product(s) are cloned into the pEGFP-C3 reporter plasmid (Clontech), which expresses the Green Fluorescence Protein (GFP), and into pcDNA3.1 (Invitrogen) which possesses the strong CMV promoter and a T7 RNA polymerase promoter and to MIGR1 retrovirus that is based on the MSCV promoter and expresses GFP under IRES control. Constructs are confirmed by sequencing.

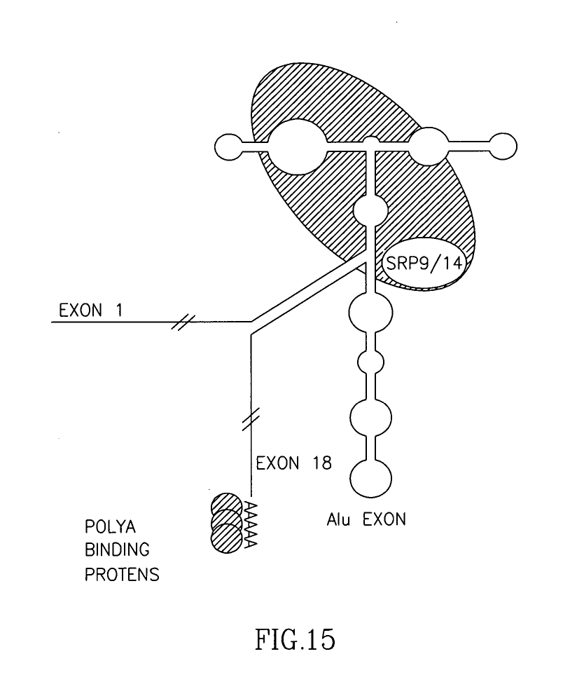

I. Association of SRP9/14 with the Alu-exon. The aim of this experiment is to check whether SRP9/14 proteins bind the Alu exon of TIF-IA mRNA in the cells. RNA folding programs predicted that the Alu exon of TIF-IA contain a putative binding site for SRP9/14 proteins (FIG. 15). To examine whether SRP9/14 binds to TIF-IA mRNA including the Alu exon, plasmids containing cDNA with or without the Alu exon are used. The plasmids are used to transfect 293T cell lines. Following 48 hr immunoprecipitation with anti-SRP9/14 antibodies, as described by Chang et al. (Nucleic Acids Res 24:4165-70, 1996). Briefly, antibodies are initially adsorbed onto protein A-Sepharose beads (Pharmacia), washed with NET-2 buffer (150 mM NaCl, 50 mM Tris-HCl, 0.05% Nonidet-P40) and then incubated with cell-derived extracts for 90 min at 4° C., washed four times with NET-2 buffer and RNA is purified by phenol/chloroform extraction and ethanol precipitation. The precipitated RNA is analyzed by RT-PCR followed by separation in agarose gel and confirmation by sequencing. In addition Northern blot analyses is carried out after hybridization to antisense [³²P] RNA probes complementary to the Alu sequence and to TIF-IA RNA.

II. The effect of TIF-IA with the Alu-exon on translation. The purpose of the in vitro transcription and translation assay is to study the possible affect of the Alu exon on translation. The hypothesis behind this experiment is that mRNA containing an Alu exon that binds SRP9/14 proteins may effect normal translation. Therefore, TIF-IA cDNA isoforms inserted into the pcDNA3.1 plasmid are incubated in the T7 coupled transcription and translation reticulocyte lysate system (Promega) in the presence of [³⁵S] methionine (Amersham). The protein products are resolved in 10% polyacrylamide SDS gel and the amount of protein produced is evaluated using a phosphorimager.

The effect of the Alu RNA on translation is examined by adding these RNAs to an in vitro translation system and exploring the level of translation of several reported genes. To investigate the effect of Alu RNA on the expression of a reporter gene in vitro, Alu is cloned in the pGEMluc plasmid containing the luciferase gene driven by the SP6 promoter. The construct is used as substrates for coupled in vitro transcription/translation using a rabbit reticulosyte lysate translation system supplemented with cytoplasmic extracts from Human HeLa cells. Luciferase activity is determined using a luminometer, and luciferase protein levels are determined by denaturing gel analysis of translation products. UV crosslinking experiments are carried out for determining the differences in binding of SRP proteins in the cytoplasmic extracts to Alu RNA according to the protocols of Malca et al. (Mol Cell Biol 23:3442-55, 2003).

Cytoplasmic extracts of HeLa cells are incubated with a labeled transcript. RNase A and T1 are added and proteins are resolved in SDS-polyacrlamide gels. An excess of unlabeled specific or non-specific Alu RNA are added to determine specificity of the protein binding.

III. The effect of T1F-IA with Alu exon on cell growth. Previous studies have shown that different regions of TIF-IA have a functional significant activity on polymerase I transcription and on cell growth (Zhao et al., Mol Cell 11:405-13, 2003). In order to test whether the Alu exon of TIF-IA enhances or abrogates TIF-IA activity thus positively or negatively effecting cell cycle or growth, a Fluorescence Activated Cell Sorter (FACS) analysis is performed. To test this, 293T and HeLa cells are transfected with cDNAs encoding both isoforms of TIF-IA co-expressed with the GFP protein. At several time points (24, 36, 48, and 60 hr) after transfection cells are stained with propidium iodide (0.05 mg/ml) and analyzed by dual-parameter flow cytometry (FACS). Initially, cells expressing GFP-tagged TIF-IA are identified and sub-grouped. Then, this group is used to measure the DNA content for cell cycle analysis. More than 50,000 events are recorded from each sample. The number of cells expressing any TIF-IA isoform and the percentage of each cell cycle stage is calculated. The results indicate that cells transfected with TIF-IA cDNA with the Alu exon are mostly in the S phase.

Because HeLa and 293T cells are of a different cell lineage than lymphoblastic leukemia similar experiments on the mouse IL3 dependent pre-B lymphoblastic cell line Baf-3 is carried out. This system is ideal for testing pro-survival factors since the cells undergo rapid and complete apoptosis upon withdrawal of IL3. Both isoforms of TIF-IA are expressed by these cells through retroviral infection. GFP positive cells are sorted and analyzed for cell cycle and growth properties. Growth factor dependency is tested by measuring proliferation and apoptosis in the presence of low concentration of IL3. Apoptosis is measured by FACS analysis using annexin positive- and propidium iodide negative-cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated polynucleotide comprising at least one Alu-derived exon comprising a sequence derived by alternative splicing of an Alu element, the Alu element comprising at least one mutated splice site as set forth in any one of SEQ ID NOS:1-58.

2. The isolated polynucleotide of claim 1, the Alu-derived exon comprising a sequence downstream to the 3′ splice-site as set forth in positions 12 to 16 of SEQ ID NOS:1-32.

3. The isolated polynucleotide of claim 1, the Alu-derived exon comprising a sequence upstream to the 5′ splice-site as set forth in positions 13 to 19 of SEQ ID NOS:33-58.

4. An oligonucleotide comprising at least a portion of an Alu-derived exon, the Alu-derived exon comprising a sequence derived by alternative splicing of an Alu element, the Alu element comprising at least one mutated splice site as set forth in any one of SEQ ID NOS:1-58, analogs and homologs thereof.

5. The oligonucleotide of claim 4 wherein the sequence comprising the mutated splice site of the Alu element comprises a sequence having at least 85% to 95% or more homology to any one of SEQ ID NOS:1-58.

6. The oligonucleotide of claim 4 wherein the oligonucleotide is selected from an oligonucleotide probe and an oligonucleotide primer.

7. The oligonucleotide of claim 4, further comprising a detectable label.

8. The oligonucleotide of claim 7, wherein the detectable label is selected from the group consisting of: a fluorophore, a chromophore, a radioactive isotope, an electron dense reagent, an enzyme, an enzymatic substrate and a ligand.

9. A construct comprising the oligonucleotide according to claim 4.

10. A vector comprising the oligonucleotide according to claim 4.

11. A vector comprising an isolated polynucleotide according to claim 1.

12. A pharmaceutical composition comprising a therapeutically effective amount of the oligonucleotide according to claim 4.

13. The pharmaceutical composition of claim 12, wherein the oligonucleotide is selected from the group consisting of: antisense nucleotide sequence, sense nucleotide sequence, short interfering RNA, ribozyme and aptamer.

14. A method for diagnosing, detecting or predicting a disease, comprising determining the presence in a biological specimen of a polynucleotide comprising a sequence selected from: a) an Alu-derived exon comprising a sequence derived by alternative splicing of an Alu element, the Alu element comprising at least one mutated splice site as set forth in any one of SEQ ID NOS:1-58; and b) an alternatively spliced mRNA isoform comprising an Alu-derived exon comprising a sequence derived by alternative splicing of an Alu element, the Alu element comprising at least one mutated splice site as set forth in any one of SEQ ID NOS:1-58.

15. The method of claim 14, wherein the Alu-derived exon comprises a sequence downstream to the 3′ splice-site as set forth in positions 12 to 16 of SEQ ID NOS:1-32.

16. The method of claim 14, wherein the Alu-derived exon comprises a sequence upstream to the 5′ splice-site as set forth in positions 13 to 19 of SEQ ID NOS:33-58.

17. The method of claim 14, wherein the disease is selected from the group consisting of: cancer, a genetic disease, a chromosomal abnormality, a genetic predisposition, and a viral infection.

18. The method of claim 14, wherein the genetic disease is selected from the group consisting of: Alport syndrome, Sly syndrome, CCFDN (congenital cataracts, facial dysmorphism, and neuropathy) syndrome and OAT deficiency.

19. The method of claim 17, wherein cancer is selected from the group consisting of: prostate cancer, breast cancer, ovarian cancer, lung cancer, melanoma, renal cancer, bladder cancer, fibrosarcoma, hepatocellular carcinoma, osteocarcinoma, primary ductal carcinoma, giant cell sarcoma, ductal carcinoma, Hodgkin's disease, colorectal carcinoma, lymphoma, transitional cell carcinoma, uterine sarcoma, adenocarcinoma, plasmacytoma, epidermoid carcinoma, Burkitt's lymphoma, Ewing's sarcoma, gastric carcinoma, squamous cell carcinoma, neuroblastoma and rhabdomyosarcoma.

20. The method of claim 14 wherein determining the presence of the polynucleotide is selected from the group consisting of in-situ hybridization, Southern blot analysis, Northern blot analysis, polymerase chain reaction, RNase protection, and selective hybridization to arrayed cDNA libraries.

21. The method of claim 14, wherein the alternatively spliced mRNA isoform is an ACAD-9 mRNA.

22. The method of claim 21 wherein the disease is ovarian cancer.

23. The method of claim 14, wherein the alternatively spliced mRNA isoform is TIF-IA mRNA.

24. The method of claim 23, wherein the disease is lymphoma.

25. The method according to claim 14, further comprising determining the presence in a biological specimen of at least one control gene or fragments thereof.

26. The method of claim 25, the at least one control gene is selected from the group consisting of: β-actin, glycer-aldehyde-phophate-dehydrogenase (GAPDH), S16 rRNA, phosphoribosyl transferase (HPRT), dihydrofolate reductase (DHFR), adenosine deaminase, phosphoglycerol kinase (PGK), pyruvate kinase, phosphoglycerol mutase.

27. The method of claim 14, wherein the biological specimen is derived from a human or non-human primate.

28. The method of claim 27, wherein the biological specimen is selected from a tissue sample, fluid, cells, cellular extract, tissue extract and genetic material extracted therefrom.

29. The method of claim 14 wherein the polynucleotide comprising the alternatively spliced mRNA isoform comprising an Alu-derived exon is selected by in silico screening of a database of sequences selected from the group consisting of: cDNA, EST, and mRNA.

30. A method for preventing or inhibiting a disease in a subject in need thereof, comprising treating the subject in need thereof with a therapeutically effective amount of a pharmaceutical composition containing as an active ingredient an oligonucleotide according to claim 4.

31. The method of claim 30, wherein the polynucleotide is selected from the group consisting of: antisense nucleotide sequence, sense nucleotide sequence, short interfering RNA, ribozyme and aptamer.


SEQUENCE LISTING (64 sequences)

SEQ ID NO: 1 - DNA, 20 bp, Artificial Sequence
  derived in silico from adenosine deaminase 2 (ADAR2) human gene
ttttttttgagacagggtct

SEQ ID NO: 2 - DNA, 20 bp, Artificial Sequence
  derived in silico from mitochondrial transcription factor B2 (TFB2M) human gene
cttttttgagacagggtctc

SEQ ID NO: 3 - DNA, 20 bp, Artificial Sequence
  derived in silico from mevalonate kinase (MVK) human gene
tttttgagacagggtctcac

SEQ ID NO: 4 - DNA, 20 bp, Artificial Sequence
  derived in silico from the human gene encoding the MTG8 related protein (CBFA2T2)
tttttgagacagggtctcac

SEQ ID NO: 5 - DNA, 20 bp, Artificial Sequence
  derived in silico from member 9 of human acyl-Coenzyme A dehydrogenase family (NPD002)
tgagacagggtcccgctctg

SEQ ID NO: 6 - DNA, 20 bp, Artificial Sequence
  derived in silico from the human gene encoding myelin/oligodendrocyte glycoprotein
ttttttttttgagacagtgt

SEQ ID NO: 7 - DNA, 20 bp, Artificial Sequence
  derived in silico from an EST library (EST No. AB007962)
acagagtcttgctctcttac

SEQ ID NO: 8 - DNA, 20 bp, Artificial Sequence
  derived in silico from the human gene encoding prostaglandin E synthase
tattttttttggagacagag

SEQ ID NO: 9 - DNA, 20 bp, Artificial Sequence
  derived, in silico, from human decay- accelerating factor 1ab gene
tttttttgagacaggttctc

SEQ ID NO: 10 - DNA, 20 bp, Artificial Sequence
  derived in silico from the human gene encoding Serine threonine kinase 2
tttttttgagacagagtctt

SEQ ID NO: 11 - DNA, 20 bp, Artificial Sequence
  derived in silico from the human gene encoding melan-A (a Melanoma antigen recognized by T-cells 1)
ttttttcttgaaacaggaaa

SEQ ID NO: 12 - DNA, 20 bp, Artificial Sequence
  derived in silico from an EST library (EST No. HSM800877)
ttttttttgaaacagcgtct

SEQ ID NO: 13 - DNA, 18 bp, Artificial Sequence
  derived in silico from the human gene encoding Integrin beta 1
tttattttgagacagtct

SEQ ID NO: 14 - DNA, 20 bp, Artificial Sequence
  derived in silico from an EST library (EST No. Ak021447)
tcttttttttgacagagtcc

SEQ ID NO: 15 - DNA, 20 bp, Artificial Sequence
  derived in silico from a human gene encoding methyl-CpG binding domain protein 3
tttttttgtgacagagtctc

SEQ ID NO: 16 - DNA, 20 bp, Artificial Sequence
  derived in silico from the human gene encoding calponin 2 (CNN2)
tttatttattttatttatgt

SEQ ID NO: 17 - DNA, 20 bp, Artificial Sequence
  derived in silico from the human gene encoding Putative glycosyltransferase (PGT)
ttttttttgagacggagttt

SEQ ID NO: 18 - DNA, 20 bp, Artificial Sequence
  derived in silico from an EST library (EST No. U92992)
ctttttttgagatggagtct

SEQ ID NO: 19 - DNA, 20 bp, Artificial Sequence
  derived in silico from human chromosome 4 open reading frame 8 (RES4-22)
tttatttcgagatggagttt

SEQ ID NO: 20 - DNA, 20 bp, Artificial Sequence
  derived in silico from the human gene encoding Zink-finger protein ANC_2H01 (LOC51193)
ttttttttgagatggagtct

SEQ ID NO: 21 - DNA, 20 bp, Artificial Sequence
  derived in silico from an EST library (EST No. AK024074)
ttttttttgagatggagtct

SEQ ID NO: 22 - DNA, 20 bp, Artificial Sequence
  derived in silico from the human gene encoding cholinergic receptor - nicotinic alpha polypeptide 3 (CHRNA3)
ttttttttgagatggagtct

SEQ ID NO: 23 - DNA, 20 bp, Artificial Sequence
  derived in silico from the human gene encoding transmembrane protein 14A (PTD011)
ttttttttgagacgcccagg

SEQ ID NO: 24 - DNA, 20 bp, Artificial Sequence
  derived in silico from the human gene encoding Hepatocellular carcinoma-associated antigen 66 (HCA66)
tttttttttagacggagtct

SEQ ID NO: 25 - DNA, 20 bp, Artificial Sequence
  derived in silico from the human gene encoding cytochrome P450 family 3 subfamily A polypeptide (43CYP3A43)
tttttttttagacagagtct

SEQ ID NO: 26 - DNA, 20 bp, Artificial Sequence
  derived in silico from the human gene encoding lecithin-cholesterol acyltransferase (LCAT)
tttttttgtagagacagggt

SEQ ID NO: 27 - DNA, 20 bp, Artificial Sequence
  derived in silico from the human gene encoding two pore segment channel 1
ttttgttttagagatggtat

SEQ ID NO: 28 - DNA, 20 bp, Artificial Sequence
  derived in silico from the human gene encoding solute carrier family 3 (activators of dibasic and neutral amino acid transport), member 2 (SLC3A2)
ttgtttttaagacagcattt

SEQ ID NO: 29 - DNA, 20 bp, Artificial Sequence
  derived in silico from the human gene encoding Cell adhesion ligand for LFA-1 (ICAM2)
tttgttttaagacagggtct

SEQ ID NO: 30 - DNA, 20 bp, Artificial Sequence
  derived in silico from the human gene encoding collagen, type IV, alpha 3 (COL4A3)
tttttctttagatggagtct

SEQ ID NO: 31 - DNA, 20 bp, Artificial Sequence
  derived in silico from the human gene encoding glucuronidase, beta (associated with Sly syndrome)
attttttggatatgcagtct

SEQ ID NO: 32 - DNA, 20 bp, Artificial Sequence
  derived in silico from the human gene encoding ornithine aminotransferase (OAT)
ttttttttgagacagagttt

SEQ ID NO: 33 - DNA, 27 bp, Artificial Sequence
  derived in silico from the human gene encoding melan-A (Melanoma antigen recognized by T-cells 1)
tagctgggaccacaggcacgtgccacc

SEQ ID NO: 34 - DNA, 27 bp, Artificial Sequence
  derived in silico from human chromosome 4 open reading frame 8 (RES4-22)
tagttggaattacaggcaagcaaaacc

SEQ ID NO: 35 - DNA, 27 bp, Artificial Sequence
  derived in silico from the human gene encoding hematopoietic progenitor kinase (HPK1)
tagctgggattacaggcatgtgccacc

SEQ ID NO: 36 - DNA, 27 bp, Artificial Sequence
  derived in silico from adenosine deaminase 2 (ADAR2) human gene
gagctgggactacaggcatgtaccact

SEQ ID NO: 37 - DNA, 27 bp, Artificial Sequence
  derived in silico from the human gene encoding two pore segment channel 1 (KIAA1169)
cagctggaactacaggtgtgcactgtg

SEQ ID NO: 38 - DNA, 27 bp, Artificial Sequence
  derived in silico from the human gene encoding Zink-finger protein ANC_2H01 (LOC51193)
tagctggggttacaggtgtgcgccacc

SEQ ID NO: 39 - DNA, 27 bp, Artificial Sequence
  derived in silico from the gene encoding X- linked zinc finger protein (ZFX)
tagctgggattacaggtgtgcgccccc

SEQ ID NO: 40 - DNA, 27 bp, Artificial Sequence
  derived in silico from the human gene encoding mevalonate kinase (MVK)
tagctgggaccacaggtgtgcgccacc

SEQ ID NO: 41 - DNA, 27 bp, Artificial Sequence
  derived in silico from the human gene encoding prostaglandin E synthase (PTGES)
tagctgggaccacaggtgtgtatcacc

SEQ ID NO: 42 - DNA, 27 bp, Artificial Sequence
  derived in silico from an EST library (EST No. AB007962)
tagctgggactacaggtgtgtgccacc

SEQ ID NO: 43 - DNA, 27 bp, Artificial Sequence
  derived in silico from the human gene encoding ecotropic viral integration site 5 (EVI5)
tggctgggactacaggtgtgtgccatc

SEQ ID NO: 44 - DNA, 26 bp, Artificial Sequence
  derived in silico from chromosome 20 open reading frame 26 (C20orf26)
tagctggactataggtatgtgccacc

SEQ ID NO: 45 - DNA, 27 bp, Artificial Sequence
  derived in silico from an EST library (EST No. AK021447)
tagctgggattacaggtatgtgccacc

SEQ ID NO: 46 - DNA, 27 bp, Artificial Sequence
  derived in silico from the human gene breast cancer 2 (BRCA2)
tagctgggactacaggtgcgtgccacc

SEQ ID NO: 47 - DNA, 27 bp, Artificial Sequence
  derived in silico from the human gene calponin 2 (CNN2)
tagctgggactacaggtgcatgctgcc

SEQ ID NO: 48 - DNA, 27 bp, Artificial Sequence
  derived in silico from the gene of baculoviral IAP repeat-containing 3 (BIRC3)
tagctggaaatacaggtgcgtgccacc

SEQ ID NO: 49 - DNA, 27 bp, Artificial Sequence
  derived in silico from  cytochrome P450 family 3 subfamily A, polypeptide 43 (CYP3A43)
gagctgggattacaggtacacaccacc

SEQ ID NO: 50 - DNA, 27 bp, Artificial Sequence
  derived in silico from an EST library (EST No. AK021447)
tagctgggattacaggtgcccgccacc

SEQ ID NO: 51 - DNA, 27 bp, Artificial Sequence
  derived in silico from human gene encoding methyl-CpG binding domain protein 3 (MBD3)
tagctgggatttcaggtacccgtcaca

SEQ ID NO: 52 - DNA, 27 bp, Artificial Sequence
  derived in silico from the human gene encoding Cytosolic phospholipase A2 beta (PLA2G4B)
tagctgggattacaggtgcccaccacc

SEQ ID NO: 53 - DNA, 27 bp, Artificial Sequence
  derived in silico from human gene encoding breast carcinoma amplified sequence 4 isoform b (BCAS4)
tagctgggattacaggtgcgcgctacc

SEQ ID NO: 54 - DNA, 27 bp, Artificial Sequence
  derived in silico from the gene encoding cell adhesion ligand for LFA-1 (ICAM2)
tagctgggatcacaggtgagagccacc

SEQ ID NO: 55 - DNA, 27 bp, Artificial Sequence
  derived in silico from the gene encoding transglutaminase 4 (TGM4)
taaccgggattacaggtatgtgactcc

SEQ ID NO: 56 - DNA, 27 bp, Artificial Sequence
  derived in silico from the gene encoding Integrin beta 1
tacctgggattacaggtgcctgcctca

SEQ ID NO: 57 - DNA, 27 bp, Artificial Sequence
  derived in silico from the gene encoding the cholinergic receptor nicotinic alpha polypeptide 3 (CHRNA3)
tgtctgggactacaggtacccgcccgc

SEQ ID NO: 58 - DNA, 27 bp, Artificial Sequence
  derived in silico from the gene encoding the carboxy-terminal domain of RNA polymerase II polypeptide A phosphatase, subunit 1
gtgttgggattacaggtatgagccatt

SEQ ID NO: 59 - DNA, 18 bp, Artificial Sequence
  Forward PCR primer used to amplify the mRNA of transcriptional intermediary factor 1 (TIF-1A)
gtcgcgttagttcggccc

SEQ ID NO: 60 - DNA, 21 bp, Artificial Sequence
  Reverse primer PCR used to amplify the mRNA of transcriptional intermediary factor 1 (TIF-1A)
cttcagcaagacttctgtcac

SEQ ID NO: 61 - DNA, 21 bp, Artificial Sequence
  Forward PCR primer used to amplify the mRNA of acyl-Coenzyme A dehydrogenase family, member 9 (ACAD-9)
gcactacatcctcaatggctc

SEQ ID NO: 62 - DNA, 22 bp, Artificial Sequence
  Reverse PCR primer used to amplify the mRNA of acyl-Coenzyme A dehydrogenase family, member 9 (ACAD-9)
cactgatccatcagaatcaacg

SEQ ID NO: 63 - DNA, 76 bp, Artificial Sequence
  Alu-derived exon obtained in silico from the gene encoding acyl-Coenzyme A dehydrogenase family, member 9
ggtcccgctctgtcacccaggctggagtgcagtggcacgatcttggctcactgcagcctcaacttcctgggctcag

SEQ ID NO: 64 - DNA, 171 bp, Artificial Sequence
  Alu-derived exon obtained in silico from the gene encoding acyl-Coenzyme A dehydrogenase family, member 9
taatttctcttttcttttgagacagggtcccgctctgtcacccaggctggagtgcagtggcacgatcttggctcactgcagcctcaacttcctgggctcaggtgattctcctacctcaacctcccaagcagctgggagtacaggcacacagctaactctttgtattttttt